SEQUENCE LISTING (56 sequences)

SEQ ID NO: 1 - DNA, 1895 bp, Homo sapiens
  CDS (18)...(311): CXCL1
gctttccagccccaaccatgcataaaaggggttcgcggatctcggagagccacagagcccgggccgcaggcacctcctcgccagctcttccgctcctctcacagccgccagacccgcctgctgagccccatggcccgcgctgctctctccgccgcccccagcaatccccggctcctgcgagtggcactgctgctcctgctcctggtagccgctggccggcgcgcagcaggtgggtaccggcgccctggggtccccgggccggacgcggctggggtaggcacccagcgccgacagcctcgctcagtcagtgagtctcttcttccctaggagcgtccgtggccactgaactgcgctgccagtgcttgcagaccctgcagggaattcaccccaagaacatccaaagtgtgaacgtgaagtcccccggaccccactgcgcccaaaccgaagtcatgtaagtcccgccccgcgctgcctctgccaccgccggggtcccagaccctcctgctgccccaaccctgtccccagcccgacctcctgcctcacgagattcccttccctctgcagagccacactcaagaatgggcggaaagcttgcctcaatcctgcatcccccatagttaagaaaatcatcgaaaagatgctgaacaggtgagttatggtttccatgtacacaggcgactggagccgttggtcagaaatactggcatgtgccccctaaaaataaaatcaggaaaacccaggggttagttgaaggactagaaattgggattattgttttcacaattaaggtttcctttacgataattactgctctggtgccagaggatattcccaatgcctggcgtccccaccctggttcttccttcgttccaatgaatgtaggtaaaactgccttcatttgaggcccagtaggacaaacagcaacaggttctggctgtttttaatccaatagtacagtggagaccaccgccccaccccacccccattcctaaaagagcatcccaagcttagaggtccctgccacacagcacagctgtcataggcagtagccacttggttgccaggctggggaaactgcattcggagaactctagaggctggaggagcagggcaggagaagagtgttgtgcaatcagctttcccgagcacctactcagggcacccattttctcattgcagtgacaaatccaactgaccagaagggaggaggaagctcactggtggctgttcctgaaggaggccctgcccttataggaacagaagaggaaagagagacacagctgcagaggccacctggattgtgcctaatgtgtttgagcatcgcttaggagaagtcttctatttatttatttattcattagttttgaagattctatgttaatattttaggtgtaaaataattaagggtatgattaactctacctgcacactgtcctattatattcattctttttgaaatgtcaaccccaagttagttcaatctggattcatatttaatttgaaggtagaatgttttcaaatgttctccagtcattatgttaatatttctgaggagcctgcaacatgccagccactgtgatagaggctggcggatccaagcaaatggccaatgagatcattgtgaaggcaggggaatgtatgtgcacatctgttttgtaactgtttagatgaatgtcagttgttatttattgaaatgatttcacagtgtgtggtcaacatttctcatgttgaaactttaagaactaaaatgttctaaatatcccttggacattttatgtctttcttgtaaggcatactgccttgtttaatggtagttttacagtgtttctggcttagaacaaaggggcttaattattgatgttttcatagagaatataaaaataaagcacttatag

SEQ ID NO: 2 - protein, 97 aa, Homo sapiens
MHKRGSRISESHRARAAGTSSPALPLLSQPPDPPAEPHGPRCSLRRPQQSPAPASGTAAPAPGSRWPARSRWVPAPWGPRAGRGWGRHPAPTASLSQ

SEQ ID NO: 3 - DNA, 1110 bp, Homo sapiens
  CDS (75)...(398): CXCL2
gacagagcccgggccacggagctccttgccagctctcctcctcgcacagccgctcgaaccgcctgctgagccccatggcccgcgccacgctctccgccgcccccagcaatccccggctcctgcgggtggcgctgctgctcctgctcctggtggccgccagccggcgcgcagcaggagcgcccctggccactgaactgcgctgccagtgcttgcagaccctgcagggaattcacctcaagaacatccaaagtgtgaaggtgaagtcccccggaccccactgcgcccaaaccgaagtcatagccacactcaagaatgggcagaaagcttgtctcaaccccgcatcgcccatggttaagaaaatcatcgaaaagatgctgaaaaatggcaaatccaactgaccagaaggaaggaggaagcttattggtggctgttcctgaaggaggccctgccttacaggaacagaagaggaaagagagacacagctgcagaggccacctggcttgcgcctaatgtgtttgagcatacttaggagaagtcttctatttatttatttatttatttatttgtttgttttagaagattctatgttaatattttatgtgtaaaataaggttatgattgaatctacttgcacactctcccattatatttattgtttattttaggtcaaacccaagttagttcaatcctgattcatatttaatttgaagatagaaggtttgcagatattctctagtcatttgttaatatttcttcgtgatgacatatcacatgtcagccactgtgatagaggctgaggaatccaagaaaatggccagtaagatcaatgtgacggcagggaaatgtatgtgtgtctattttgtaactgtaaagatgaatgtcagttgttatttattgaaatgatttcacagtgtgtggtcaacatttctcatgttgaagctttaagaactaaaatgttctaaatatcccttggcattttatgtctttcttgtaagatactgccttgtttaatgttaattatgcagtgtttccctctgtgttagagcagagaggtttcgatatttattgatgttttcacaaagaacaggaaaataaaatatttaaaaatat

SEQ ID NO: 4 - protein, 107 aa, Homo sapiens
MARATLSAAPSNPRLLRVALLLLLLVAASRRAAGAPLATELRCQCLQTLQGIHLKNIQSVKVKSPGPHCAQTEVIATLKNGQKACLNPASPMVKKIIEKMLKNGKSN

SEQ ID NO: 5 - DNA, 1064 bp, Homo sapiens
  CDS (78)...(398): CXCL3
cacagccgggtcgcaggcacctccccngccagctctcccgcattctgcacagcttcccgacgcgtctgctgagccccatggcccacgccacgctctccgccgcccccagcaatccccggctcctgcgggtggcgctgctgctcctgctcctggtgggcagccggcgcgcagcaggagcgtccgtggtcactgaactgcgctgccagtgcttgcagacactgcagggaattcacctcaagaacatccaaagtgtgaatgtaaggtcccccggaccccactgcgcccaaaccgaagtcatagccacactcaagaatgggaagaaagcttgtctcaaccccgcatcccccatggttcagaaaatcatcgaaaagatactgaacaaggggagcaccaactgacaggagagaagtaagaagcttatcagcgtatcattgacacttcctgcagggtggtccctgcccttaccagagctgaaaatgaaaaagagaacagcagctttctagggacagctggaaagggacttaatgtgtttgactatttcttacgagggttctacttatttatgtatttatttttgaaagcttgtattttaatattttacatgctgttatttaaagatgtgagtgtgtttcatcaaacatagctcagtcctgattatttaattggaatatgatgggttttaaatgtgtcattaaactaatatttagtgggagaccataatgtgtcagccaccttgataaatgacagggtggggaactggagggtngggggattgaaatgcaagcaattagtggatcactgttagggtaagggaatgtatgtacacatctattttttatacttttttttttaaaaaagaatgtcagttgttatttattcaaattatctcacattatgtgttcaacatttttatgctgaagtttcccttagacattttatgtcttgcttgtagggcataatgccttgtttaatgtccattctgcagcgtttctctttcccttggaaaagagaatttatcattactgttacatttgtacaaatgacatgataataaaagttttatg

SEQ ID NO: 6 - protein, 106 aa, Homo sapiens
MAHATLSAAPSNPRLLRVALLLLLLVGSRRAAGASVVTELRCQCLQTLQGIHLKNIQSVNVRSPGPHCAQTEVIATLKNGKKACLNPASPMVQKIIEKILNKGSTN

SEQ ID NO: 7 - DNA, 439 bp, Homo sapiens
  CDS (8)...(304): CXCL4
ccgcagcatgagctccgcagccgggttctgcgcctcacgccccgggctgctgttcctggggttgctgctcctgccacttgtggtcgccttcgccagcgctgaagctgaagaagatggggacctgcagtgcctgtgtgtgaagaccacctcccaggtccgtcccaggcacatcaccagcctggaggtgatcaaggccggaccccactgccccactgcccaactgatagccacgctgaagaatggaaggaaaatttgcttggacctgcaagccccgctgtacaagaaaataattaagaaacttttggagagttagctactagctgcctacgtgtgtgcatttgctatatagcatacttcttttttccagtttcaatctaactgtgaaagaaacttctgatatttgtgttatccttatgattttaaataaacaaaataaatc

SEQ ID NO: 8 - protein, 99 aa, Homo sapiens
MSSAAGFCASRPGLLFLGLLLLPLVVAFASAEAEEDGDLQCLCVKTTSQVRPRHITSLEVIKAGPHCPTAQLIATLKNGRKICLDLQAPLYKKIIKKLL

SEQ ID NO: 9 - DNA, 1173 bp, Homo sapiens
  CDS (107)...(451): CXCL5
cggcacgagcacagtgctccggatcctccaatcttcgctcctccaatctccgctcctccacccagttcaggaacccgcgaccgctcgcagcgctctcttgaccactatgagcctcctgtccagccgcgcggcccgtgtccccggtccttcgagctccttgtgcgcgctgttggtgctgctgctgctgctgacgcagccagggcccatcgccagcgctggtcctgccgctgctgtgttgagagagctgcgttgcgtttgtttacagaccacgcagggagttcatcccaaaatgatcagtaatctgcaagtgttcgccataggcccacagtgctccaaggtggaagtggtagcctccctgaagaacgggaaggaaatttgtcttgatccagaagccccttttctaaagaaagtcatccagaaaattttggacggtggaaacaaggaaaactgattaagagaaatgagcacgcatggaaaagtttcccagtctacagcagagaagttttctggaggtctctgaacccagggaagacaagaaggaaagattttgttgttgtttgtttatttggtttccccagtagttagctttcttccctggattcctcacttttgaagagtgtgaggaaaacctatgtttggcgcttaagctttcagctcagcttaatgaagtgtttagcatagtacctctgctatttgctgttattttatctgctatgctattgaagttttggcaattgactatagtgtgagccaggaatcactggctgttaatcttacaaagtgtcttggaattgtaggtgactattatttttccaagaaatatcccttaagatattaactgagaaggctgggggtttaatgtggaaatgatgtttcaaaaggaatcctgtgatggaaatacaactggtatcttcacttttttaggaattgggaaatattttaatgtttcttggggaatatgttagagaattcccttactcttgattgtgggatactatttaattatttcactttagaaagctgagtgtttcacaccttatctatgtagaatatatttccttattcagaatttctaaaagtttaagttctatgagggctaatatcttatcttcctataattttagacattgctttaactttttagtaaaaaaaaaaaaaaaaaaaaaaaaaaaa

SEQ ID NO: 10 - protein, 114 aa, Homo sapiens
MSLLSSRAARVPGPSSSLCALLVLLLLLTQPGPIASAGPAAAVLRELRCVCLQTTQGVHPKMISNLQVFAIGPQCSKVEVVASLKNGKEICLDPEAPFLKKVIQKILDGGNKEN

SEQ ID NO: 11 - DNA, 673 bp, Homo sapiens
  CDS (67)...(453): CXCL7
gggcaactcaccctcactcagaggtcttctggttctggaaacaactctagctcagccttctccaccatgagcctcagacttgataccaccccttcctgtaacagtgcgagaccacttcatgccttgcaggtgctgctgcttctgtcattgctgctgactgctctggcttcctccaccaaaggacaaactaagagaaacttggcgaaaggcaaagaggaaagtctagacagtgacttgtatgctgaactccgctgcatgtgtataaagacaacctctggaattcatcccaaaaacatccaaagtttggaagtgatcgggaaaggaacccattgcaaccaagtcgaagtgatagccacactgaaggatgggaggaaaatctgcctggacccagatgctcccagaatcaagaaaattgtacagaaaaaattggcaggtgatgaatctgctgattaatttgttctgtttctgccaaacttctttaactcccaggaagggtagaattttgaaaccttgattttctagagttctcatttattcaggatacctattcttactgtattaaaatttggatatgtgtttcattctgtctcaaaaatcacattttattctgagaaggttggttaaaagatggcagaaagaagatgaaaataaataagcctggtttcaaccctct

SEQ ID NO: 12 - protein, 128 aa, Homo sapiens
MSLRLDTTPSCNSARPLHALQVLLLLSLLLTALASSTKGQTKRNLAKGKEESLDSDLYAELRCMCIKTTSGIHPKNIQSLEVIGKGTHCNQVEVIATLKDGRKICLDPDAPRIKKIVQKKLAGDESAD

SEQ ID NO: 13 - DNA, 1639 bp, Homo sapiens
  CDS (91)...(390): CXCL8
acaaactttcagagacagcagagcacacaagcttctaggacaagagccaggaagaaaccaccggaaggaaccatctcactgtgtgtaaacatgacttccaagctggccgtggctctcttggcagccttcctgatttctgcagctctgtgtgaaggtgcagttttgccaaggagtgctaaagaacttagatgtcagtgcataaagacatactccaaacctttccaccccaaatttatcaaagaactgagagtgattgagagtggaccacactgcgccaacacagaaattattgtaaagctttctgatggaagagagctctgtctggaccccaaggaaaactgggtgcagagggttgtggagaagtttttgaagagggctgagaattcataaaaaaattcattctctgtggtatccaagaatcagtgaagatgccagtgaaacttcaagcaaatctacttcaacacttcatgtattgtgtgggtctgttgtagggttgccagatgcaatacaagattcctggttaaatttgaatttcagtaaacaatgaatagtttttcattgtaccatgaaatatccagaacatacttatatgtaaagtattatttatttgaatctacaaaaaacaacaaataatttttgaatataaggattttcctagatattgcacgggagaatatacaaatagcaaaattgggccaagggccaagagaatatccgaactttaatttcaggaattgaatgggtttgctagaatgtgatatttgaagcatcacataaaaatgatgggacaataaattttgccataaagtcaaatttagctggaaatcctggatttttttctgttaaatctggcaaccctagtctgctagccaggatccacaagtccttgttccactgtgccttggtttctcctttatttctaagtggaaaaagtattagccaccatcttacctcacagtgatgttgtgaggacatgtggaagcactttaagttttttcatcataacataaattattttcaagtgtaacttattaacctatttattatttatgtatttatttaagcatcaaatatttgtgcaagaatttggaaaaatagaagatgaatcattgattgaatagttataaagatgttatagtaaatttattttattttagatattaaatgatgttttattagataaatttcaatcagggtttttagattaaacaaacaaacaattgggtacccagttaaattttcatttcagataaacaacaaataattttttagtataagtacattattgtttatctgaaattttaattgaactaacaatcctagtttgatactcccagtcttgtcattgccagctgtgttggtagtgctgtgttgaattacggaataatgagttagaactattaaaacagccaaaactccacagtcaatattagtaatttcttgctggttgaaacttgtttattatgtacaaatagattcttataatattatttaaatgactgcatttttaaatacaaggctttatatttttaactttaagatgtttttatgtgctctccaaattttttttactgtttctgattgtatggaaatataaaagtaaatatgaaacatttaaaatataatttgttgtcaaagt

SEQ ID NO: 14 - protein, 99 aa, Homo sapiens
MTSKLAVALLAAFLISAALCEGAVLPRSAKELRCQCIKTYSKPFHPKFIKELRVIESGPHCANTEIIVKLSDGRELCLDPKENWVQRVVEKFLKRAENS

SEQ ID NO: 15 - DNA, 2545 bp, Homo sapiens
  CDS (40)...(417): CXCL9
atccaatacaggagtgacttggaactccattctatcactatgaagaaaagtggtgttcttttcctcttgggcatcatcttgctggttctgattggagtgcaaggaaccccagtagtgagaaagggtcgctgttcctgcatcagcaccaaccaagggactatccacctacaatccttgaaagaccttaaacaatttgccccaagcccttcctgcgagaaaattgaaatcattgctacactgaagaatggagttcaaacatgtctaaacccagattcagcagatgtgaaggaactgattaaaaagtgggagaaacaggtcagccaaaagaaaaagcaaaagaatgggaaaaaacatcaaaaaaagaaagttctgaaagttcgaaaatctcaacgttctcgtcaaaagaagactacataagagaccacttcaccaataagtattctgtgttaaaaatgttctattttaattataccgctatcattccaaaggaggatggcatataatacaaaggcttattaatttgactagaaaatttaaaacattactctgaaattgtaactaaagttagaaagttgattttaagaatccaaacgttaagaattgttaaaggctatgattgtctttgttcttctaccacccaccagttgaatttcatcatgcttaaggccatgattttagcaatacccatgtctacacagatgttcacccaaccacatcccactcacaacagctgcctggaagagcagccctaggcttccacgtactgcagcctccagagagtatctgaggcacatgtcagcaagtcctaagcctgttagcatgctggtgagccaagcagtttgaaattgagctggacctcaccaagctgctgtggccatcaacctctgtatttgaatcagcctacaggcctcacacacaatgtgtctgagagattcatgctgattgttattgggtatcaccactggagatcaccagtgtgtggctttcagagcctcctttctggctttggaagccatgtgattccatcttgcccgctcaggctgaccactttatttctttttgttcccctttgcttcattcaagtcagctcttctccatcctaccacaatgcagtgcctttcttctctccagtgcacctgtcatatgctctgatttatctgagtcaactcctttctcatcttgtccccaacaccccacagaagtgctttcttctcccaattcatcctcactcagtccagcttagttcaagtcctgcctcttaaataaacctttttggacacacaaattatcttaaaactcctgtttcacttggttcagtaccacatgggtgaacactcaatggttaactaattcttgggtgtttatcctatctctccaaccagattgtcagctccttgagggcaagagccacagtatatttccctgtttcttccacagtgcctaataatactgtggaactaggttttaataattttttaattgatgttgttatgggcaggatggcaaccagaccattgtctcagagcaggtgctggctctttcctggctactccatgttggctagcctctggtaacctcttacttattatcttcaggacactcactacagggaccagggatgatgcaacatccttgtctttttatgacaggatgtttgctcagcttctccaacaataagaagcacgtggtaaaacacttgcggatattctggactgtttttaaaaaatatacagtttaccgaaaatcatataatcttacaatgaaaaggactttatagatcagccagtgaccaaccttttcccaaccatacaaaaattccttttcccgaaggaaaagggctttctcaataagcctcagctttctaagatctaacaagatagccaccgagatccttatcgaaactcattttaggcaaatatgagttttattgtccgtttacttgtttcagagtttgtattgtgattatcaattaccacaccatctcccatgaagaaagggaacggtgaagtactaagcgctagaggaagcagccaagtcggttagtggaagcatgattggtgcccagttagcctctgcaggatgtggaaacctccttccaggggaggttcagtgaattgtgtaggagaggttgtctgtggccagaatttaaacctatactcactttcccaaattgaatcactgctcacactgctgatgatttagagtgctgtccggtggagatcccacccgaacgtcttatctaatcatgaaactccctagttccttcatgtaacttccctgaaaaatctaagtgtttcataaatttgagagtctgtgacccacttaccttgcatctcacaggtagacagtatataactaacaaccaaagactacatattgtcactgacacacacgttataatcatttatcatatatatacatacatgcatacactctcaaagcaaataatttttcacttcaaaacagtattgacttgtataccttgtaatttgaaatattttctttgttaaaatagaatggtatcaataaatagaccattaatcag

SEQ ID NO: 16 - protein, 125 aa, Homo sapiens
MKKSGVLFLLGIILLVLIGVQGTPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTT

SEQ ID NO: 17 - DNA, 1172 bp, Homo sapiens
  CDS (67)...(363): CXCL10
gagacattcctcaattgcttagacatattctgagcctacagcagaggaacctccagtctcagcaccatgaatcaaactgcgattctgatttgctgccttatctttctgactctaagtggcattcaaggagtacctctctctagaaccgtacgctgtacctgcatcagcattagtaatcaacctgttaatccaaggtctttagaaaaacttgaaattattcctgcaagccaattttgtccacgtgttgagatcattgctacaatgaaaaagaagggtgagaagagatgtctgaatccagaatcgaaggccatcaagaatttactgaaagcagttagcaaggaaatgtctaaaagatctccttaaaaccagaggggagcaaaatcgatgcagtgcttccaaggatggaccacacagaggctgcctctcccatcacttccctacatggagtatatgtcaagccataattgttcttagtttgcagttacactaaaaggtgaccaatgatggtcaccaaatcagctgctactactcctgtaggaaggttaatgttcatcatcctaagctattcagtaataactctaccctggcactataatgtaagctctactgaggtgctatgttcttagtggatgttctgaccctgcttcaaatatttccctcacctttcccatcttccaagggtactaaggaatctttctgctttggggtttatcagaattctcagaatctcaaataactaaaaggtatgcaatcaaatctgctttttaaagaatgctctttacttcatggacttccactgccatcctcccaaggggcccaaattctttcagtggctacctacatacaattccaaacacatacaggaaggtagaaatatctgaaaatgtatgtgtaagtattcttatttaatgaaagactgtacaaagtataagtcttagatgtatatatttcctatattgttttcagtgtacatggaataacatgtaattaagtactatgtatcaatgagtaacaggaaaattttaaaaatacagatagatatatgctctgcatgttacataagataaatgtgctgaatggttttcaaataaaaatgaggtactctcctggaaatattaagaaagactatctaaatgttgaaagatcaaaaggttaataaagtaattataact

SEQ ID NO: 18 - protein, 98 aa, Homo sapiens
MNQTAILICCLIFLTLSGIQGVPLSRTVRCTCISISNQPVNPRSLEKLEIIPASQFCPRVEIIATMKKKGEKRCLNPESKAIKNLLKAVSKEMSKRSP

SEQ ID NO: 19 - DNA, 1371 bp, Homo sapiens
  CDS (85)...(354): CXCL11
ctccttccaagaagagcagcaaagctgaagtagcagcaacagcaccagcagcaacagcaaaaaacaaacatgagtgtgaagggcatggctatagccttggctgtgatattgtgtgctacagttgttcaaggcttccccatgttcaaaagaggacgctgtctttgcataggccctggggtaaaagcagtgaaagtggcagatattgagaaagcctccataatgtacccaagtaacaactgtgacaaaatagaagtgattattaccctgaaagaaaataaaggacaacgatgcctaaatcccaaatcgaagcaagcaaggcttataatcaaaaaagttgaaagaaagaatttttaaaaatatcaaaacatatgaagtcctggaaaagggcatctgaaaaacctagaacaagtttaactgtgactactgaaatgacaagaattctacagtaggaaactgagacttttctatggttttgtgactttcaacttttgtacagttatgtgaaggatgaaaggtgggtgaaaggaccaaaaacagaaatacagtcttcctgaatgaatgacaatcagaattccactgcccaaaggagtccagcaattaaatggatttctaggaaaagctaccttaagaaaggctggttaccatcggagtttacaaagtgctttcacgttcttacttgttgtattatacattcatgcatttctaggctagagaaccttctagatttgatgcttacaactattctgttgtgactatgagaacatttctgtctctagaagttatctgtctgtattgatctttatgctatattactatctgtggttacagtggagacattgacattattactggagtcaagcccttataagtcaaaagcatctatgtgtcgtaaagcattcctcaaacattttttcatgcaaatacacayttctttccccaaatatcatgtagcacatcaatatgtagggaaacattcttatgcatcatttggtttgttttataaccaattcattaaatgtaattcataaaatgtactatgaaaaaaattatacgctatgggatactggcaacagtgcacatatttcataaccaaattagcagcaccggtcttaatttgatgtttttcaacttttattcattgagatgttttgaagcaattaggatatgtgtgtttactgtactttttgttttgatccgtttgtataaatgatagcaatatcttggacacatttgaaatacaaaatgtttttgtctaccaaagaaaaatgttgaaaaataagcaaatgtatacctagcaatcacttttactttttgtaattctgtctcttagaaaaatacataatctaatcaaaaaaaaaaaaaaaaaaaaaa

SEQ ID NO: 20 - protein, 89 aa, Homo sapiens [sequence omitted]

SEQ ID NO: 21 - DNA, 1216 bp, Homo sapiens
  CDS (19)...(72): CXCL13
ttcggcacttgggagaagatgtttgaaaaaactgactctgctaatgagcctggactcagagctcaagtctgaactctacctccagacagaatgaagttcatctcgacatctctgcttctcatgctgctggtcagcagcctctctccagtccaaggtgttctggaggtctattacacaagcttgaggtgtagatgtgtccaagagagctcagtctttatccctagacgcttcattgatcgaattcaaatcttgccccgtgggaatggttgtccaagaaaagaaatcatagtctggaagaagaacaagtcaattgtgtgtgtggaccctcaagctgaatggatacaaagaatgatggaagtattgagaaaaagaagttcttcaactctaccagttccagtgtttaagagaaagattccctgatgctgatatttccactaagaacacctgcattcttcccttatccctgctctggattttagttttgtgcttagttaaatcttttccagggagaaagaacttccccatacaaataaggcatgaggactatgtgaaaaataaccttgcaggagctgatggggcaaactcaagcttcttcactcacagcaccctatatacacttggagtttgcattcttattcatcagggaggaaagtttctttgaaaatagttattcagttataagtaatacaggattattttgattatatacttgttgtttaatgtttaaaatttcttagaaaacaatggaatgagaatttaagcctcaaatttgaacatgtggcttgaattaagaagaaaattatggcatatattaaaagcaggcttctatgaaagactcaaaaagctgcctgggaggcagatggaacttgagcctgtcaagaggcaaaggaatccatgtagtagatatcctctgcttaaaaactcactacggaggagaattaagtcctacttttaaagaatttctttataaaatttactgtctaagattaatagcattcgaagatccccagacttcatagaatactcagggaaagcatttaaagggtgatgtacacatgtatcctttcacacatttgccttgacaaacttctttcactcacatctttttcactgactttttttgtgggggcggggccggggggactctggtatctaattctttaatgattcctataaatctaatgacattcaataaagttgagcaaacattttactt

SEQ ID NO: 22 - protein, 17 aa, Homo sapiens
MFEKTDSANEPGLRAQV

SEQ ID NO: 23 - DNA, 2376 bp, Homo sapiens
  CDS (28)...(1503): PBEF
cgcgcggcccctgtcctccggcccgagatgaatcctgcggcagaagccgagttcaacatcctcctggccaccgactcctacaaggttactcactataaacaatatccacccaacacaagcaaagtttattcctactttgaatgccgtgaaaagaagacagaaaactccaaattaaggaaggtgaaatatgaggaaacagtattttatgggttgcagtacattcttaataagtacttaaaaggtaaagtagtaaccaaagagaaaatccaggaagccaaagatgtctacaaagaacatttccaagatgatgtctttaatgaaaagggatggaactacattcttgagaagtatgatgggcatcttccaatagaaataaaagctgttcctgagggctttgtcattcccagaggaaatgttctcttcacggtggaaaacacagatccagagtgttactggcttacaaattggattgagactattcttgttcagtcctggtatccaatcacagtggccacaaattctagagagcagaagaaaatattggccaaatatttgttagaaacttctggtaacttagatggtctggaatacaagttacatgattttggctacagaggagtctcttcccaagagactgctggcataggagcatctgctcacttggttaacttcaaaggaacagatacagtagcaggacttgctctaattaaaaaatattatggaacgaaagatcctgttccaggctattctgttccagcagcagaacacagtaccataacagcttgggggaaagaccatgaaaaagatgcttttgaacatattgtaacacagttttcatcagtgcctgtatctgtggtcagcgatagctatgacatttataatgcgtgtgagaaaatatggggtgaagatctaagacatttaatagtatcgagaagtacacaggcaccactaataatcagacctgattctggaaaccctcttgacactgtgttaaaggttttggagattttaggtaagaagtttcctgttactgagaactcaaagggttacaagttgctgccaccttatcttagagttattcaaggggatggagtagatattaataccttacaagagattgtagaaggcatgaaacaaaaaatgtggagtattgaaaatattgccttcggttctggtggaggtttgctacagaagttgacaagagatctcttgaattgttccttcaagtgtagctatgttgtaactaatggccttgggattaacgtcttcaaggacccagttgctgatcccaacaaaaggtccaaaaagggccgattatctttacataggacgccagcagggaattttgttacactggaggaaggaaaaggagaccttgaggaatatggtcaggatcttctccatactgtcttcaagaatggcaaggtgacaaaaagctattcatttgatgaaataagaaaaaatgcacagctgaatattgaactggaagcagcacatcattaggctttatgactgggtgtgtgttgtgtgtatgtaatacataatgtttattgtacagatgtgtggggtttgtgttttatgatacattacagccaaattatttgttggtttatggacatactgccctttcattttttttcttttccagtgtttaggtgatctcaaattaggaaatgcatttaaccatgtaaaagatgagtgctaaagtaagctttttagggccctttgccaataggtagtcattcaatctggtattgatcttttcacaaataacagaactgagaaacttttatatataactgatgatcacataaaacagatttgcataaaattaccatgattgctttatgtttatatttaacttgtatttttgtacaaacaagattgtgtaagatatatttgaagtttcagtgatttaacagtctttccaacttttcatgatttttatgagcacagactttcaagaaaatacttgaaaataaattacattgccttttgtccattaatcagcaaataaaacatggccttaacaaagttgtttgtgttattgtacaatttgaaaattatgtcgggacataccctatagaattactaaccttactgccccttgtagaatatgtattaatcattctacattaaagaaaataatggttcttactggaatgtctaggcactgtacagttattatatatcttggttgttgtattgtaccagtgaaatgccaaatttgaaaggcctgtactgcaattttatatgtcagagattgcctgtggctctaatatgcacctcaagattttaaggagataatgtttttagagagaatttctgcttccactatagaatatatacataaatgtaaaatacttacaaaagtgg

SEQ ID NO: 24 - protein, 491 aa, Homo sapiens [sequence omitted]

SEQ ID NO: 25 - DNA, 1011 bp, Homo sapiens
  CDS (15)...(131): CCL2
ggtttattttccagatgcaatcaatgccccagtcacctgctgttataacttcaccaataggaagatctcagtgcagaggctcgcgagctatagaagaatcaccagcagcaagtgtcccaaacaagctgtgatgtgagttcagcacaccaaccttccctggcctgaagttcttccttgtggagcaagggacaagcctcataaacctagagtcagagagtgcactatttaacttaatgtacaaaggttcccaatgggaaaactgaggcaccaagggaaaaagtgaaccccaacatcactctccacctgggtgcctattcagaacacccaatttctttagcttgaagtcaggatggctccacctggacacctataggagcagtttgccctgggttccctccttccacctgcgttcctcctctagctcccatggcagccctttggtgcagaatgggctgcacttctagaccaaaactgcaaaggaacttcatctaactctgtcctccctccccacagcttacagaccattgtggcaaggagatctgtgctgaccccaagcagaagtgggttcaggattccatggaccacctggacaagcaaacccaaactccgaagacttgaacactcactccacaacccaagaatctgcagctaacttatttttccctagctttccccagacaccttgtttattttattataatgaattttgtttgttgatgtgaaacattatgccttaagtaatgttaattcttatttaagttattgatgttttaagtttatctttcatggtactagtgttttttagatacagagacttggggaaattgcttttcctcttgaaccacagttctacccctgggatgttttgagggtctttgcaagaatcattaatacaaagaattttttttaacattccaatgcattgctaaaatattattgtggaaatgaatattttgtaactattacaccaaataaatatatttttgtacaaaaaaaaaaaaaaaaaaaaaa

SEQ ID NO: 26 - protein, 38 aa, Homo sapiens [sequence omitted]

SEQ ID NO: 27 - DNA, 1412 bp, Homo sapiens
  CDS (160)...(1362): PHLDA1
ctccttgggcgatcgcctgggtagaggagaggagtttccggggctcgggtccgggtcgccttccaggggaacgagcgcggaagcaagtgggcggcgagaggcggagcaagagacgctggagggcgtggacgcagcgggctttggaaaggccccaagttaatgaggcgtgcgccggctgccgagcgcctcttggagctgggctttcccccgcggtgcgggcgccaggagccgccttttccgctgggtgtcactcgggggtggggaagatggcccattcaaaagcgccgcgagggggcccggccagtgcccttcagtgagcgctcgcaagaggacggcagaggcccggcagctcggagctccgggaccttgtggcgcatcaggacgcggctgtccctctgccgggacccagagccgccgccgccgctctgcctcctgcgtgttagcctcctctgcgcgctccgggcaggcggccgtgggagccgctggggcgaggacggcgcgaggctgctgctgctgcccccggcccgcgcggctggaaacggagaggccgagccaagcggcggcccctcttatgctgggaggatgctggagagtagcggctgcaaagcgctgaaggagggcgtgctggagaagcgcagcgacgggttgttgcagctctggaagaaaaagtgttgcatcctcaccgaggaagggctgctgcttatcccgcccaagcagctgcaacaccagcagcagcagcaacagcagcagcagcagcagcaacaacagcccgggcaggggccggccgagccgtcccaacccagtggccccgctgtcgccagcctcgagccgccggtcaagctcaaggaactgcacttctccaacatgaagaccgtggactgtgtggagcgcaagggcaagtacatgtacttcactgtggtgatggcagagggcaaggagatcgactttcggtgcccgcaagaccagggctggaacgccgagatcacgctgcagatggtgcagtacaagaatcgtcaggccatcctggcggtcaaatccacgcggcagaagcagcagcacctggtccagcagcagcccccctcgcagccgcagccgcagccgcagctccagccccaaccccagcctcagcctcagccgcaaccccagccccaatcacaaccccagcctcagccccaacccaagcctcagccccagcagctccacccgtatccgcatccacatccacatccacactctcatcctcactcgcacccacaccctcacccgcacccgcatccgcaccaaataccgcacccacacccacagccgcactcgcagccgcacgggcaccggcttctccgcagcacctccaactctgcctgaaaggggcagctcccgggcaagacaaggttttgaggacttgaggaagtggg

SEQ ID NO: 28 - protein, 400 aa, Homo sapiens
MRRAPAAERLLELGFPPRCGRQEPPFPLGVTRGWGRWPIQKRREGARPVPFSERSQEDGRGPAARSSGTLWRIRTRLSLCRDPEPPPPLCLLRVSLLCALRAGGRGSRWGEDGARLLLLPPARAAGNGEAEPSGGPSYAGRMLESSGCKALKEGVLEKRSDGLLQLWKKKCCILTEEGLLLIPPKQLQHQQQQQQQQQQQQQQPGQGPAEPSQPSGPAVASLEPPVKLKELHFSNMKTVDCVERKGKYMYFTVVMAEGKEIDFRCPQDQGWNAEITLQMVQYKNRQAILAVKSTRQKQQHLVQQQPPSQPQPQPQLQPQPQPQPQPQPQPQSQPQPQPQPKPQPQQLHPYPHPHPHPHSHPHSHPHPHPHPHPHQIPHPHPQPHSQPHGHRLLRSTSNSA

SEQ ID NO: 29 - DNA, 1900 bp, Homo sapiens
  CDS (73)...(1320): SERPINB2
acaactctcagaggagcattgcccgtcagacagcaactcagagaataaccagagaacaaccagattgaaacaatggaggatctttgtgtggcaaacacactctttgccctcaatttattcaagcatctggcaaaagcaagccccacccagaacctcttcctctccccatggagcatctcgtccaccatggccatggtctacatgggctccaggggcagcaccgaagaccagatggccaaggtgcttcagtttaatgaagtgggagccaatgcagttacccccatgactccagagaactttaccagctgtgggttcatgcagcagatccagaagggtagttatcctgatgcgattttgcaggcacaagctgcagataaaatccattcatccttccgctctctcagctctgcaatcaatgcatccacagggaattatttactggaaagtgtcaataagctgtttggtgagaagtctgcgagcttccgggaagaatatattcgactctgtcagaaatattactcctcagaaccccaggcagtagacttcctagaatgtgcagaagaagctagaaaaaagattaattcctgggtcaagactcaaaccaaaggcaaaatcccaaacttgttacctgaaggttctgtagatggggataccaggatggtcctggtgaatgctgtctacttcaaaggaaagtggaaaactccatttgagaagaaactaaatgggctttatcctttccgtgtaaactcggctcagcgcacacctgtacagatgatgtacttgcgtgaaaagctaaacattggatacatagaagacctaaaggctcagattctagaactcccatatgctggagatgttagcatgttcttgttgcttccagatgaaattgccgatgtgtccactggcttggagctgctggaaagtgaaataacctatgacaaactcaacaagtggaccagcaaagacaaaatggctgaagatgaagttgaggtatacataccccagttcaaattagaagagcattatgaactcagatccattctgagaagcatgggcatggaggacgccttcaacaagggacgggccaatttctcagggatgtcggagaggaatgacctgtttctttctgaagtgttccaccaagccatggtggatgtgaatgaggagggcactgaagcagccgctggcacaggaggtgttatgacagggagaactggacatggaggcccacagtttgtggcagatcatccttttctttttcttattatgcataagataaccaactgcattttatttttcggcagattttcctcaccctaaaactaagcgtgctgcttctgcaaaagatttttgtagatgagctgtgtgcctcagaattgctatttcaaattgccaaaaatttagagatgttttctacatatttctgctcttctgaacaacttctgctacccactaaataaaaacacagaaataattagacaattgtctattataacatgacaaccctattaatcatttggtcttctaaaatgggatcatgcccatttagattttccttactatcagtttatttttataacattaacttttactttgttatttattattttatataatggtgagtttttaaattattgctcactgcctatttaatgtagctaataaagttatagaagcagatgatctgttaatttcctatctaataaatgcctttaattgttctcataatgaagaataagtaggtaccctccatgcccttctgtaataaatatctggaaaaaacattaaacaataggcaaatatatgttatgtgcatttctagaaatacataacacatatatatgtctgtatcttatattcaattgcaagtatataataaataaacctgcttccaaacaac

SEQ ID NO: 30 - protein, 415 aa, Homo sapiens
MEDLCVANTLFALNLFKHLAKASPTQNLFLSPWSISSTMAMVYMGSRGSTEDQMAKVLQFNEVGANAVTPMTPENFTSCGFMQQIQKGSYPDAILQAQAADKIHSSFRSLSSAINASTGNYLLESVNKLFGEKSASFREEYIRLCQKYYSSEPQAVDFLECAEEARKKINSWVKTQTKGKIPNLLPEGSVDGDTRMVLVNAVYFKGKWKTPFEKKLNGLYPFRVNSAQRTPVQMMYLREKLNIGYIEDLKAQILELPYAGDVSMFLLLPDEIADVSTGLELLESEITYDKLNKWTSKDKMAEDEVEVYIPQFKLEEHYELRSILRSMGMEDAFNKGRANFSGMSERNDLFLSEVFHQAMVDVNEEGTEAAAGTGGVMTGRTGHGGPQFVADHPFLFLIMHKITNCILFFGRFSSP

SEQ ID NO: 31 - DNA, 1562 bp, Homo sapiens
  CDS (312)...(1130): TGIF
ctggaattcggggcgccgagcaggagcagggaacaaaggagcggagaggggaggggagagagttgggcgagggagagcccccggccggctgccagaagatcctggcgggaggaagcccaagtgtcacttgaattccacccaaggagcgggcgcctgggatcagagcgtcctgtttagcaataacggctggagcacgtcctacaagttacgggagagtcggctgtgaaggagacgttcgcttatcccctgtgtccccgctcctggcccctccagacccccgccttgcctcgcgctgggaggggagatccagaatgaaaggcaagaaaggtattgttgcagcatctggcagtgagactgaggatgaggacagcatggacattcccttggacctttcttcatccgctggctcaggcaagagaaggagaaggggcaacctacccaaggagtctgtgcagattcttcgggattggctgtatgagcaccgttacaatgcctatccttcagagcaagaaaaagcgttgctgtcccagcaaacacacctgtctacgctacaggtctgtaactggttcatcaacgcccgccgcaggctcctccctgacatgctgagaaaggatggcaaagatccaaatcagttcacaatttcccgccgtggggccaagatttctgaaacgagctctgtggagtccgtgatgggcatcaaaaacttcatgccagctctagaggagaccccatttcattcctgtacagctgggccaaacccaaccctagggaggccactgtctcctaagccgtcatccccgggatcagttttggctcgtccatcagtgatctgccataccactgtgactgcattgaaagatgtccctttctctctctgccagtcggtcggtgtgggacaaaacacagatatacagcagatagcggccaaaaacttcacagacacctctctcatgtacccagaggacacttgtaaatctggaccaagtacgaatacacagagtggtcttttcaacactcctccccctactccaccggacctcaaccaggacttcagtggatttcagcttctagtggatgttgcactcaaacgggctgcagagatggagcttcaggcaaaacttacagcttaacccattttcaagcaaaacagttctcagaaatgtcatgattgccggggtgaaggcaagagatgaattgcattattttatatattttttattaatatttgcacatgggattgctaaaacagcttcctgttactgagatgtcttcaatggaatacagtcattccaagaactataaacttaaagctactgtagaaacaaagggttttcttttttaaatgtttcttggtagattattcataatgtgagatggttcccaatatcatgtgatttttttttttcctccccttccctttttttgttattttttcagactgtgcaatacttagagaacctatagcatcttctcattcccatgtggaacaggatgcccacatactgtctaattaataaattttccattttttttcaaacaagtaaaaaaaaaaaaaaaaaaaa

SEQ ID NO: 32 - protein, 272 aa, Homo sapiens
MKGKKGIVAASGSETEDEDSMDIPLDLSSSAGSGKRRRRGNLPKESVQILRDWLYEHRYNAYPSEQEKALLSQQTHLSTLQVCNWFINARRRLLPDMLRKDGKDPNQFTISRRGAKISETSSVESVMGIKNFMPALEETPFHSCTAGPNPTLGRPLSPKPSSPGSVLARPSVICHTTVTALKDVPFSLCQSVGVGQNTDIQQIAAKNFTDTSLMYPEDTCKSGPSTNTQSGLFNTPPPTPPDLNQDFSGFQLLVDVALKRAAEMELQAKLTA

SEQ ID NO: 33 - DNA, 4212 bp, Homo sapiens
  CDS (41)...(352): THBD
cttgcaatccaggctttccttggaagtggctgtaacatgtatgaaaagaaagaaaggaggaccaagagatgaaagagggctgcacgcgtgggggcccgagtggtgggcggggacagtcgtcttgttacaggggtgctggccttccctggcgcctgcccctgtcggccccgcccgagaacctccctgcgccagggcagggtttactcatcccggcgaggtgatcccatgcgcgagggcgggcgcaagggcggccagagaacccagcaatccgagtatgcggcatcagcccttcccaccaggcacttccttccttttcccgaacgtccagggagggagggccgggcacttataaactcgagccctggccgatccgcatgtcagaggctgcctcgcaggggctgcgcgcacggcaagaagtgtctgggctgggacggacaggagaggctgtcgccatcggcgtcctgtgcccctctgctccggcacggccctgtcgcagtgcccgcgctttccccggcgcctgcacgcggcgcgcctgggtaacatgcttggggtcctggtccttggcgcgctggccctggccggcctggggttccccgcacccgcagagccgcagccgggtggcagccagtgcgtcgagcacgactgcttcgcgctctacccgggccccgcgaccttcctcaatgccagtcagatctgcgacggactgcggggccacctaatgacagtgcgctcctcggtggctgccgatgtcatttccttgctactgaacggcgacggcggcgttggccgccggcgcctctggatcggcctgcagctgccacccggctgcggcgaccccaagcgcctcgggcccctgcgcggcttccagtgggttacgggagacaacaacaccagctatagcaggtgggcacggctcgacctcaatggggctcccctctgcggcccgttgtgcgtcgctgtctccgctgctgaggccactgtgcccagcgagccgatctgggaggagcagcagtgcgaagtgaaggccgatggcttcctctgcgagttccacttcccagccacctgcaggccactggctgtggagcccggcgccgcggctgccgccgtctcgatcacctacggcaccccgttcgcggcccgcggagcggacttccaggcgctgccggtgggcagctccgccgcggtggctcccctcggcttacagctaatgtgcaccgcgccgcccggagcggtccaggggcactgggccagggaggcgccgggcgcttgggactgcagcgtggagaacggcggctgcgagcacgcgtgcaatgcgatccctggggctccccgctgccagtgcccagccggcgccgccctgcaggcagacgggcgctcctgcaccgcatccgcgacgcagtcctgcaacgacctctgcgagcacttctgcgttcccaaccccgaccagccgggctcctactcgtgcatgtgcgagaccggctaccggctggcggccgaccaacaccggtgcgaggacgtggatgactgcatactggagcccagtccgtgtccgcagcgctgtgtcaacacacagggtggcttcgagtgccactgctaccctaactacgacctggtggacggcgagtgtgtggagcccgtggacccgtgcttcagagccaactgcgagtaccagtgccagcccctgaaccaaactagctacctctgcgtctgcgccgagggcttcgcgcccattccccacgagccgcacaggtgccagatgttttgcaaccagactgcctgtccagccgactgcgaccccaacacccaggctagctgtgagtgccctgaaggctacatcctggacgacggtttcatctgcacggacatcgacgagtgcgaaaacggcggcttctgctccggggtgtgccacaacctccccggtaccttcgagtgcatctgcgggcccgactcggcccttgcccgccacattggcaccgactgtgactccggcaaggtggacggtggcgacagcggctctggcgagcccccgcccagcccgacgcccggctccaccttgactcctccggccgtggggctcgtgcattcgggcttgctcataggcatctccatcgcgagcctgtgcctggtggtggcgcttttggcgctcctctgccacctgcgcaagaagcagggcgccgccagggccaagatggagtacaagtgcgcggccccttccaaggaggtagtgctgcagcacgtgcggaccgagcggacgccgcagagactctgagcggcctccgtccaggagcctggctccgtccaggagctgtgcctcctcacccccagctttgctaccaaagcaccttagctggcattacagctggagaagaccctccccgcaccccccaagctgttttcttctattccatggctaactggcgagggggtgattagagggaggagaatgagcctcggcctcttccgtgacgtcactggaccactgggcaatgatggcaattttgtaacgaagacacagactgcgatttgtcccaggtcctcactaccgggcgcaggagggtgagcgttattggtcggcagccttctgggcagaccttgacctcgtgggctagggatgactaaaatatttattttttttaagtatttaggtttttgtttgtttcctttgttcttacctgtatgtctccagtatccactttgcacagctctccggtctctctctctctacaaactcccacttgtcatgtgacaggtaaactatcttggtgaatttttttttcctagccctctcacatttatgaagcaagccccacttattccccattcttcctagttttctcctcccaggaactgggccaactcacctgagtcaccctacctgtgcctgaccctacttcttttgctcatctagctgtctgctcagacagaacccctacatgaaacagaaacaaaaacactaaaaataaaaatggccatttgctttttcaccagatttgctaatttatcctgaaatttcagattcccagagcaaaataattttaaacaaagggttgagatgtaaaaggtattaaattgatgttgctggactgtcatagaaattacacccaaagaggtatttatctttacttttaaacagtgagcctgaattttgttgctgttttgatttgtactgaaaaatggtaattgttgctaatcttcttatgcaatttccttttttgttattattacttatttttgacagtgttgaaaatgttcagaaggttgctctagattgagagaagagacaaacacctcccaggagacagttcaagaaagcttcaaactgcatgattcatgccaattagcaattgactgtcactgttccttgtcactggtagaccaaaataaaaccagctctactggtcttgtggaattgggagcttgggaatggatcctggaggatgcccaattagggcctagccttaatcaggtcctcagagaatttctaccatttcagagaggccttttggaatgtggcccctgaacaagaattggaagctgccctgcccatgggagctggttagaaatgcagaatcctaggctccaccccatccagttcatgagaatctatatttaacaagatctgcagggggtgtgtctgctcagtaatttgaggacaaccattccagactgcttccaattttctggaatacatgaaatatagatcagttataagtagcaggccaagtcaggcccttattttcaagaaactgaggaattttctttgtgtagctttgctctttggtagaaaaggctaggtacacagctctagacactgccacacagggtctgcaaggtctttggttcagctaagctaggaatgaaatcctgcttcagtgtatggaaataaatgtatcatagaaatgtaacttttgtaagacaaaggttttcctcttctattttgtaaactcaaaatatttgtacatagttatttatttattggagataatctagaacacaggcaaaatccttgcttatgacatcacttgtacaaaataaacaaataacaatgtgctctcggtgtgtgtctgttcacttttcctccctcagtgccctgcatttatgtcattaaatgcgggctcacaaaccatgcaaatgctatgagatgcatggagggctgccctgtaccccagcacttctattgtctggtgatggcaccatctctgatctttcaaa

SEQ ID NO: 34 - protein, 103 aa, Homo sapiens
MKRKKGGPRDERGLHAWGPEWWAGTVVLLQGCWPSLAPAPVGPAREPPCARAGFTHPGEVIPCARAGARAAREPSNPSMRHQPFPPGTSFLFPNVQGGRAGHL

SEQ ID NO: 35 - DNA, 1230 bp, Homo sapiens
  CDS (210)...(968): AREG
agacgttcgcacacctgggtgccagcgccccagaggtcccgggacagcccgaggcgccgcgcccgccgccccgagctccccaagccttcgagagcggcgcacactcccggtctccactcgctcttccaacacccgctcgttttgcggcagctcgtgtcccagagaccgagttgccccagagaccgagacgccgccgctgcgaaggaccaatgagagccccgctgctaccgccggcgccggtggtgctgtcgctcttgatactcggctcaggccattatgctgctggattggacctcaatgacacctactctgggaagcgtgaaccattttctggggaccacagtgctgatggatttgaggttacctcaagaagtgagatgtcttcagggagtgagatttcccctgtgagtgaaatgccttctagtagtgaaccgtcctcgggagccgactatgactactcagaagagtatgataacgaaccacaaatacctggctatattgtcgatgattcagtcagagttgaacaggtagttaagcccccccaaaacaagacggaaagtgaaaatacttcagataaacccaaaagaaagaaaaagggaggcaaaaatggaaaaaatagaagaaacagaaagaagaaaaatccatgtaatgcagaatttcaaaatttctgcattcacggagaatgcaaatatatagagcacctggaagcagtaacatgcaaatgtcagcaagaatatttcggtgaacggtgtggggaaaagtccatgaaaactcacagcatgattgacagtagtttatcaaaaattgcattagcagccatagctgcctttatgtctgctgtgatcctcacagctgttgctgttattacagtccagcttagaagacaatacgtcaggaaatatgaaggagaagctgaggaacgaaagaaacttcgacaagagaatggaaatgtacatgctatagcataactgaagataaaattacaggatatcacattggagtcactgccaagtcatagccataaatgatgagtcggtcctctttccagtggatcataagacaatggaccctttttgttatgatggttttaaactttcaattgtcactttttatgctatttctgtatataaaggtgcacgaaggtaaaaagtattttttcaagttgtaaataatttatttaatatttaatggaagtgtatttattttacagctcattaaacttttttaacc

SEQ ID NO: 36 - protein, 252 aa, Homo sapiens
MRAPLLPPAPVVLSLLILGSGHYAAGLDLNDTYSGKREPFSGDHSADGFEVTSRSEMSSGSEISPVSEMPSSSEPSSGADYDYSEEYDNEPQIPGYIVDDSVRVEQVVKPPQNKTESENTSDKPKRKKKGGKNGKNRRNRKKKNPCNAEFQNFCIHGECKYIEHLEAVTCKCQQEYFGERCGEKSMKTHSMIDSSLSKIALAAIAAFMSAVILTAVAVITVQLRRQYVRKYEGEAEERKKLRQENGNVHAIA

SEQ ID NO: 37 - DNA, 439 bp, Artificial Sequence
  Probe sequence
attaactctacctgcacactgtcctattatattcattctttttgaaatgtcaaccccaagttagttcaatctggattcatatttaatttgaaggtagaatgttttcaaatgttctccagtcattatgttaatatttctgaggagcctgcaacatgccagccactgtgatagaggctggcggatccaagcaaatggccaatgagatcattgtgaaggcaggggaatgtatgtgcacatctgttttgtaactgtttagatgaatgtcagttgttatttattgaaatgatttcacagtgtgtggtcaacatttctcatgttgaaactttaagaactaaaatgttctaaatatcccttggacattttatgtctttcttgtaaggcatactgccttgtttaatggtagttttacagtgtttctggcttagaacaaaggggctta

SEQ ID NO: 38 - DNA, 444 bp, Artificial Sequence
  Probe sequence
cgcctaatgtgtttgagcatcacttaggagaagtcntctatttatttatttattnattnatttgttngttttagaagnttctatgttaatattttatgtgtaaaataaggttatgantgaatctacttgcacactcncccattatatttattgtttattttaggtcaaacccaagttagttcaatcctgattcatatttaatttgaagatagaaggtttgcagatattctctagtcatttgttaatatttcttcgtgatgacatatcacatgtcagccactgtgatagaggctgaggaatccaagaaaatggccagtnagatcaatgtgannnnnnnnnnnnnnnnnnnngtctattttgtaactgtaaagatgaatgtcagttgttatttattgaaatgatttcacagtgtgtggtcaacatttctcatgttgaagctttaag

SEQ ID NO: 39 - DNA, 569 bp, Artificial Sequence
  Probe sequence
gtatcattgacacttcctgcagggtggtccctngccctnacnaganctgaaantgaaaangagancagcagctttctagggacagctggaaanggacttaatgtgtttgactatttcttacgagggttctacttatttatgtatttatttttgaaagcttgtattttaatattttacatgctgttatttaaagatgtgagtgtgtttcatcaaacatagctcagtcctgattatttaattggaatatgatgggttttaaatgtgtcattaaactaatatttagtgggagaccataatgtgtcagccaccttgataaatgacagggtggggaactggagggtggggggattgaaatgcaagcaattagtggatcactgttagggtaagggaatgtatgtacacatctattttttatactttttttttaaaaaaagaatgtcagttgttatttattcaaattatctcacattatgtgttcaacatttttatgctgaagtttcccttagacattttatgtcttgcttgtagggcataatgccttgtttaatgtccattctgcagcgtttctc

SEQ ID NO: 40 - DNA, 344 bp, Artificial Sequence
  Probe sequence
ccccgggctgctgttcctggggttgctgctcctgccacttgtggtcgccttcgccagcgctgaagctgaagaagatggggacctgcagtgcctgtgtgtgaagaccacctcccaggtccgtcccaggcacatcaccagcctggaggtgatcaaggccggaccccactgccccactgcccaactgatagccacgctgaagaatggaaggaaaatttgcttggacctgcaagccccgctgtacaagaaaataattaagaaacttttggagagttagctactagctgcctacgtgtgtgcatttgctatatagcatacttcttttttccagtttcaatctaactgtg

SEQ ID NO: 41 - DNA, 545 bp, Artificial Sequence
  Probe sequence
tttctggaggtctctgaacccagggaagacaagaaggaaagattttgttgttgtttgtttatttggtttccccagtagttagctttcttccctggattcctcacttttgaagagtgtgaggaaaacctatgtttggcgcttaagctttcagctcagcttaatgaagtgtttagcatagtacctctgctatttgctgttattttatctgctatgctattgaagtttnnnnnnnnnnnnnnnnnnnnnnnnnnnnnnnnnnnnnnnntaatcttacaaagtgtcttggaattgtaggtgactattatttttccaagaaatatcccttaagatattaactgagaaggctgggggtttaatgtggaaatgatgtttcaaaaggaatcctgtgatggaaatacaactggtatcttcacttttttaggaattgggaaatattttaatgtttcttggggaatatgttagagaattcccttactcttgattgtgggatactatttaattatttcactttagaaagctgagtgtttcacaccttatctatgtag

SEQ ID NO: 42 - DNA, 542 bp, Artificial Sequence
  Probe sequence
gctctggcttcctccaccaaaggacaaactaagagaaacttggncgaaaggcaaagaggaaagtctagacagtgacttgtatgctgaacnccgctgncatgtgtatnaaagnacaaccntctgggaattncatcccnaaaaacatccnnaaagtttggnaagtgatcggggaaagggaacccnattggcaaccnaagtcngaagtnnatagnccacantgaaggatgggnaggnaaatntgccnggnccccagatgntncccagaatncaagaaaattgtnacagnaaaaattnnggcaggtngatgnatctgctgattannntnnnnnnnnnnnnnnnnnnnnnnnnnaactcccaggaagggtagaattttgaaaccttgattttctagagttctcatttattcaggatacctattcttactgtattaaaatttggatatgtgtttcattctgtctcaaaaatcacattttattctgagaaggttggttaaaagatggcagaaagaagatgaaaataaataagcctggtttcaaccctct

SEQ ID NO: 43 - DNA, 386 bp, Artificial Sequence
  Probe sequence
tatttgtgcaagaatttggaaaaatagaagatgaatcattgattgaatagttataaagatgttatagtaantttattttattttagatattaaatgatgttttattagataaatttcaatcagggtttttagattaaacaaacaaacaattgggtacccagttaaattttcatttcagatanacaacaaataattttttagtataagtacattattgtttatctgaaattttaattgaactaacaatcctagtttgatactcccagtcttnnnnnnnnnnnnnnngttggtagtgctgtgttgaattacggaataatgagttagaactattaaaacagccaaaactccacagtcaatattagtaatttcttgctggttgaaacttg

SEQ ID NO: 44 - DNA, 575 bp, Artificial Sequence
  Probe sequence
ccaagtcggttagtggaagcatgattggtgcccagttagcctctgcaggatgtggaaacctccttccaggggaggttcagtgaattgtgtaggagaggttgtctgtggccagaatttaaacctatactcactttcccaaattgaatcacngctcacactgctgatgatttagagtgnnnnnnnnnnnnnnnnnnacccgaacgtcttatctaatcatgaaactccctagttccttcatgtaacttccctgaaaaatctaagtgtttcataaatttgagagtctgtgacccacttaccttgcatctcacaggtagacagtatataactaacaaccaaagactacatattgtcactgacacacacgttataatcatttatcatatatatacatacatgcatacactctcaaagcaaataatttttcacttcaaaacagtattgacttgtataccttgtaatttgaaatattttctttgttaaaatagaatggtatcaataaatagaccattaatcagaaaanaaaaaaaaaaaaannnnctcttgaatgtacccttcaactgttgaatgtttaatag

SEQ ID NO: 45 - DNA, 235 bp, Artificial Sequence
  Probe sequence
actctaagtggcattcaaggagtacctctctctagaaccgtacgctgtacctgcatcagcattagtaatcaacctgttaatccaaggtctttagaaaaacttgaaattattcctgcaagccaattttgtccacgtgttgagatcattgctacaatgaaaaagaagggtgagaagagatgtctgaatccagaatcgaaggccatcaagaatttactgaaagcagttagcaaggaaa

SEQ ID NO: 46 - DNA, 384 bp, Artificial Sequence
  Probe sequence
ggagtcaagcccttataagtcaaaagcatctatgtgtcgtaaagcattcctcaaacattttttcatgcaaatacacanttctttccccaaatatcatgtagcacatcaatatgtagggaaacattcttatgcatcatttggtttgttttataaccaattcattaaatgtaattcataaaatgtactatgaaaaaaattatacgctatgggatactggcaacagtgcacatatttcataaccaaattagcagcaccggtcttaatttgatgtttttcaacttttattcattgagatgttttgaagcaattaggatatgtgtgtttactgtactttttgttttgatccgtttgtataaatgatagcaatatcttggacacatttga

SEQ ID NO: 47 - DNA, 403 bp, Artificial Sequence
  Probe sequence
cctcaaatttgaacatgtggcttgaattaagaagaaaattatggcatatattaaaagcaggcttctatgaaagactcaaaaagctgcctgggaggcagatggaacttgagcctgtcaagaggcaaaggaatccatgtagtagatatcctctgcttaaaaactcactacggaggagaattaagtcctacttttaaagaatttctttataaaatttactgtctaagattaatagcattcgaagatccccagacttcatagaatactcagggaaagcatttaaagggtgatgtacacatgtatcctttcacacatttgccttgacaaacttctttcactcacatctttttcactgactttttttgtggggggcggggccggggggactctggtatctaattcttta

SEQ ID NO: 48 - DNA, 606 bp, Artificial Sequence
  Probe sequence
tcttactggaatgtctaggcactgtacagttattatatatcttggttgttgtattgtaccagtgaaatgccaaatttgaaaggcctgtactgcaattttatatgtcagagattgcctgtggctctaatatgcacctcaagattttaaggagataatgtttttagagagaatttctgcttccactatagaatatatacataaatgtaaaatacttacaaaagtggaagtagtgtattttaaagtaattacacttctgaatttatttttcatattctatagttggtatgacttaaatgaattactggagtgggtagtgagtgtacttaaatgtttcaattctgttatattttttattaagtttttaaaaaattaaattggatattaaattgtannnanannannnantaattttaaactgaatgccctcaataagtaatactgaagnnnnttcttaaatgaagataaattatctccaatgaaaagcatgacatgtgtttcaatagaagaatcttaagttggctaaattcaaagtgcttgacatcaaaatgttctngagtgattagctactagattctgaatcatacatcacatctgactagagaccag

SEQ ID NO: 49 - DNA, 325 bp, Artificial Sequence
  Probe sequence
tgggttcaggattccatggaccacctggacaagcaaacccaaactccgaagacttgaacactcactccacaacccaagaatctgcagctaacttatttttccctagctttccccagacaccttgtttattttattataatgaattttgtttgttgatgtgaaacattatgccttaagtaatgttaattcttatttaagttattgatgttttaagtttatctttcatggtactagtgttttttagatacagagacttggggaaattgcttttcctcttgaaccacagttctacccctgggatgttttgagggtctttgcaagaatc

SEQ ID NO: 50 - DNA, 397 bp, Artificial Sequence
  Probe sequence
agtacaagaatcgtcaggccatcctggcggtcaaatccacgcggcanaagcagcagcacctggtccagcagcagcccccctcgcagccgcagccgcagccgcagctccagccccaaccccagcctcagcctcagccgcaaccccagccccaatcacaaccccagcctcagccccaacccaagcctcagccccagcagctccacccgtatccgcatccacatccacatccacactctcatcctcactcgcacccacaccctcacccgcacccgcatccgcaccaaataccgcacccacacccacagccgcactcgcagccgcacgggcaccggcttctccgcagcacctccaactctgcctgaaaggggcagctcccgggcaagacaaggttttgagg

SEQ ID NO: 51 - DNA, 452 bp, Artificial Sequence
  Probe sequence
ctcaccctaaaactaagcgtgctgcttctgcaaaagatttttgtagatgagctgtgtgcctcagaattgctatttcaaattgccaaaantttannnnnnnnnnnnnnnnnnnnctgctcttctgaacaacttctgctacccactaaataaaaacacagaaataattagacaattgtctattataacatgacaaccctattaatcatttggtcttctaaaatgggatcatgcccatttagattttccttactatcagtttatttttataacattaacttttactttgttatttattattttatataatggtgagtttttaaattattgctcactgcctatttaatgtagctaataaagttatagaagcagatgatctgttaatttcctatctaataaatgcctttaattgttctcataatgaagaataagtaggtatccctccatgcccttct

SEQ ID NO: 52 - DNA, 453 bp, Artificial Sequence
  Probe sequence
tcagcttctagtggatgttgcactcaaacgggctgcagagatggagcttcaggcaaaacttacagcnttaancccattttcaagcaaaacagttctcagaaatgtcatgattgccggggtgaaggcaagagatgaattngcattattttatatattttttattaatatttgcacatgggattgctaaaacagcttcctgttactgagatgtcttcaatggaatacagtcattccaagaactataaacttaaagctactgtagaaacaaagggttttcttttttaaatgtttcttggtagattattcataatgtgagatggttcccaatatcatgtgantttttttttcctccccttccctttttttgttattttttcagactgtgcaatacttagagaacctatagcatcttctcattcccatgtggaacaggatgcccacatactgtctaat

SEQ ID NO: 53 - DNA, 528 bp, Artificial Sequence
  Probe sequence
tttctaccatttcagagaggccttttggaatgtggcccctgaacaagaattggaagctgccctgcccatgggagctggttagaaatgcagaatcctaggctccaccccatccagttcatgagaatctatatttaacaagatctgcagggggtgtgtctgctcagtaatttgaggacaaccattccnagactgcttccaattttctggaatacatgaaatatagatcagttataagtagcaggccaagtcaggcccttattttcaagaaactgaggaattttctttgtgtagctttgctctttggtagaaaaggctaggtacacagctctagacactgccacacagggtctgcaaggtctttggttcagctaagctaggaatgaaatcctgcttcagtgtatggaaataaatgtatcatagaaatgtaacttttgtaagacaaaggttttcctcttctattttgtaaactcaaaatatttgtacatagttatttatttattggagataatctagaacacaggcaaaatc

SEQ ID NO: 54 - DNA, 449 bp, Artificial Sequence
  Probe sequence
tatatagagcacctggaagcagtaacatgcaaatgtcagcaagaatatttcggtgaacggtgtgggnaaaagtccatgaaaactcacagcatgattgacagtagtttatcaaaaattgcattagcagccatagctgcctttatgtctgctgtgatcctcnnnnnnnnnnnnnnnnnnnnnnnnnnnnnnnnnnnnnnnnncgtcaggaaatatgaaggagaagctgaggaacgaaagaaacttcgacaagagaatggaaatgtacatgctatagcataactgaagataaaattacaggatatcacattggagtcantnccaagtcatagccataaatgatgagtcggtcctctttccagtggatcataagacaatggaccctttttgttatgatggttttaaactttcaattgtcactttttatgctatttctgtatataaaggtgcac

SEQ ID NO: 55 - DNA, 1723 bp, Homo sapiens
  CDS (1039)...(1686): VEGF-vascular endothelial growth factor
tcgcggaggcttggggcagccgggtagctcggaggtcgtggcgctgggggctagcaccagcgctctgtcgggaggcgcagcggttaggtggaccggtcagcggactcaccggccagggcgctcggtgctggaatttgatattcattgatccgggttttatccctcttcttttttcttaaacatttttttttaaaactgtattgtttctcgttttaatttatttttgcttgccattccccacttgaatcgggccgacggcttggggagattgctctacttccccaaatcactgtggattttggaaaccagcagaaagaggaaagaggtagcaagagctccagagagaagtcgaggaagagagagacggggtcagagagagcgcgcgggcgtgcgagcagcgaaagcgacaggggcaaagtgagtgacctgcttttgggggtgaccgccggagcgcggcgtgagccctcccccttgggatcccgcagctgaccagtcgcgctgacggacagacagacagacaccgcccccagccccagctaccacctcctccccggccggcggcggacagtggacgcggcggcgagccgcgggcaggggccggagcccgcgcccggaggcggggtggagggggtcggggctcgcggcgtcgcactgaaacttttcgtccaacttctgggctgttctcgcttcggaggagccgtggtccgcgcgggggaagccgagccgagcggagccgcgagaagtgctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagccgcgcgcgctccccaggccctggcccgggcctcgggccggggaggaagagtagctcgccgaggcgccgaggagagcgggccgccccacagcccgagccggagagggagcgcgagccgcgccggccccggtcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaaaaatcagttcgaggaaagggaaaggggcaaaaacgaaagcgcaagaaatcccggtataagtcctggagcgttccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtgagccgggcaggaggaaggagcctccctcagggtttcgg

SEQ ID NO: 56 - protein, 215 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVPCGPCSERRKHLFVQDPQTCKCSCKNTDSRCKARQLELNERTCRCDKPRR

(19) United States
(12) Patent Application Publication
(10) Pub. No.: US 2005/0164244 A1
(43) Pub. Date: Jul. 28, 2005

(54) Methods of determining juvenile arthritis classification

(76) Inventors: David Glass, Cincinnati, OH (US); Michael Barnes, Cincinnati, OH (US); Susan Thompson, Cincinnati, OH (US)

Correspondence Address: TAFT, STETTINIUS & HOLLISTER LLP, SUITE 1800, 425 WALNUT STREET, CINCINNATI, OH, 45202-3957, US

(21) Appl. No.: 10/970,698

(22) Filed: Oct. 21, 2004

Related U.S. Application Data
(60) Provisional application No. 60/513,826, filed on Oct. 23, 2003.
(60) Provisional application No. 60/517,642, filed on Nov. 5, 2003.

(51) Int. Cl. C12Q 1/68

(52) U.S. Cl. 435/6

(57) ABSTRACT

The present application is directed to a method of determining disease classification, particular of juvenile arthritis. It is also directed to a method of analyzing disease progression in a subject exhibiting juvenile arthritis. The invention pertains to expression patterns of certain inflammatory related nucleotide sequences that differ among the various classifications of juvenile arthritis such as, but not limited to, pauciarticular arthritis, polyarticular arthritis, juvenile onset spondyloarthropathy, and systemic onset juvenile rheumatoid arthritis.

### CROSS-REFERENCE TO RELATED APPLICATIONS

This application claims priority to and benefit of U.S. Provisional Patent Application Ser. Nos. 60/513,826 and 60/517,642, filed on Oct. 23, 2003 and Nov. 5, 2003 respectively, which are herein incorporated by reference in their entirety.

### FIELD OF THE INVENTION

The present invention relates to the field of differential gene expression in juvenile arthritis.

### BACKGROUND OF THE INVENTION

Chronic inflammatory arthritis is a source of morbidity for about 70,000 children in the United States alone. There are currently three classification systems for the juvenile arthritides (Petty, R. E. and Cassidy, J. T. (2001) The Juvenile Idiopathic Arthritides. In Textbook of Pediatric Rheumatology. 4^{th} ed. J. T. Cassidy and R. E. Petty, ed. W.B. Sauders Co., St. Louis p. 214, herein incorporated by reference). Juvenile rheumatoid arthritis (JRA) is defined by American College of Rheumatology criteria, while juvenile chronic arthritis (JCA) and juvenile idiopathic arthritis (JIA) correspond to European League against rheumatism and the International League of Associations for Rheumatology criteria, respectively. See Petty, R. E. and Cassidy, J. T. (2001) The Juvenile Idiopathic Arthritides. In Textbook of pediatric Rheumatology. 4^{th} ed. J. T. Cassidy and R. E. Petty, ed. W.B. Sauders Co., St. Louis p. 214; Cassidy et al. (1986) Arthritis Rheum. 29: 274, and Petty et al. (1998) J. Rheumatol. 25: 1991; herein incorporated by reference in their entirety. Despite differences in classification systems, subtypes of juvenile arthritis are generally characterized by the number of affected joints within six months of onset. Pauciarticular arthritis involves four or fewer joints, while polyarticular affects five or more joints. Systemic onset juvenile rheumatoid arthritis (SOJRA) is characterized by spiking fevers and rash, which may occur prior to the onset of clinical arthritis. Further classification and sub-classification can be based on age at onset with early onset arthritis beginning before six years of age, and late onset at six or greater. The number of affected joints beyond the first six months of disease is used to describe disease course, with pauciarticular course defined by four or fewer joints and polyarticular course defined by five or more joints. Although SOJRA may be pauciarticular at onset, it typically follows a polyarticular course. Predicting disease course for children with pauciarticular onset arthritis has not been possible.

Spondyloarthropathies, although more common in adults, can begin during childhood and may be confused with late-onset pauciarticular JRA at onset when there is an absence of enthesitis and axial involvement. The presence of enthesitis is a useful predictor for juvenile onset spondyloarthropathy (JSpA), and HLA-B27 is frequently positive. However, many children with juvenile onset spondyloarthropathy with peripheral arthritis do not go on to develop axial disease, and it has not been possible to identify those who will progress.

Cytokines are a large group of polypeptides and small proteins that are secreted by cells of the immune system. Although cytokine functions are complex, cytokine profiles are relevant parameters of an immune response. The ratio of pro- and anti-inflammatory cytokines and the T helper cell subtypes is considered important in the pathogenesis of autoimmune diseases including juvenile idiopathic arthritis. The measurement of cytokines and chemotactic cytokines in body fluids and synovial tissue has provided insight into the type of immune and inflammatory reaction. Differences between subtypes of juvenile idiopathic arthritis have been identified with these measurements. However, cytokine measurements in serum have not been useful for diagnostic purposes because of the variability of cytokines during 24 hour periods, variability in the collection and assay methods, and ease of degradation of most cytokines. See Woo, P. (2002) Curr. Rheumatol. Rep. 4: 452-457, herein incorporated by reference in its entirety.

Current juvenile arthritis classification based on the pattern of joint involvement (e.g. pauciarticular juvenile rheumatoid arthritis, polyarticular juvenile rheumatoid arthritis, or juvenile onset spondyloarthropathy) has some prognostic value. However, it still does not allow accurate identification of patients who are bound to develop severe destructive arthritis and who would benefit most from aggressive treatment started early in the disease.

Thus, development of a method of determining the classification of juvenile arthritis is desirable. It is of importance to develop a method of predicting disease outcome with respect to joint destruction. It is of importance to develop a method of determining juvenile arthritis classification that would identify subjects likely to benefit from early aggressive treatment.

### SUMMARY OF THE INVENTION

Methods and kits for determining disease classification, particularly of juvenile arthritides, and for analyzing disease progression in subjects exhibiting juvenile arthritis are provided. The inventions are based on the novel discovery that certain inflammatory related nucleotide sequences are expressed differently in the various classifications of juvenile arthritis, particularly pauciarticular juvenile arthritis, polyarticular juvenile arthritis, systemic onset juvenile rheumatoid arthritis, and juvenile onset spondyloarthropathies. The expression patterns of the nucleotide sequences of interest in peripheral blood monocytes and/or synovial fluid monocytes differ among the categories of juvenile arthritides. Methods of the invention allow determination of disease classification by analyzing the expression patterns of the nucleotide sequences of interest from various tissues in a subject. The invention further provides a method of analyzing disease progression and kits for performing the methods of the invention. Additionally, the invention provides methods of identifying expression modulating compounds and arthritis modulating compounds.

The present invention involves analyzing the expression pattern of CXCL chemokines (composed of both angiogenic and angiostatic factors) to classify juvenile arthritis in a subject, to predict the course of the juvenile arthritis, and/or to predict the efficacy of treatments. The invention is based on the discovery that certain chemokines are differentially expressed in peripheral blood monocytes (PBMC) and synovial fluid monocytes (SFMC) in various classifications of juvenile rheumatoid arthritis. These chemokines are members of a family of angiogenic and angiostatic cytokines defined by the presence or absence of an ELR amino acid motif. Gene expression analysis of peripheral blood monocytes identified angiogenic chemokines including, but not limited to, CXCL1, CXCL2, CXCL3, and CXCL8, with altered expression in polyarticular samples compared to other disease subtypes or controls. Expression of these and additional angiogenic cytokines in synovial fluid monocytes were equivalent between juvenile arthritis types. Expression of several angiostatic cytokines including, but not limited to, CXCL9, CXCL10, and CXCL11, in peripheral blood monocytes were equivalent between juvenile arthritis types Expression analysis of these and additional angiostatic cytokines indicate differing expression between the juvenile arthritis disease subtypes in synovial fluid monocytes.

In a first embodiment, the invention provides a method of determining disease classification in a subject. The method involves the steps of obtaining a peripheral blood monocyte sample, a synovial fluid monocyte sample, or both a peripheral blood monocyte sample and a synovial fluid monocyte sample, from a subject, assaying the expression level of a nucleotide sequence of interest in the samples, and comparing the expression levels of the nucleotide sequence of interest to a standard expression pattern to determine disease classification. In an aspect of the invention, the method further comprises isolating RNA from the samples. Nucleotide sequences of interest include, but are not limited to, the nucleotide sequences set forth in SEQ ID NOS:1, 3, 5, 7, 9, 11, 13, 15, 17, 19, 21, 23, 25, 27, 29, 31, 33, 35, 37, 38, 39, 40, 41, 42, 43, 44, 45, 46, 47, 48, 49, 50, 51, 52, 53, 54, and 55; nucleotide sequences encoding the amino acid sequences set forth in SEQ ID NOS:2, 4, 6, 8, 10, 12, 14, 16, 18, 20, 22, 24, 26, 28, 30, 32, 34, 36, 56, and fragments and variants thereof. Many of the nucleotide sequences of interest are CXCL chemokines. In another aspect, the expression level assay analyzes the polypeptide encoded by the nucleotide sequence of interest. In an aspect of the invention, the subject is a mammal, particularly a human. In an aspect of the invention, the subject exhibits a juvenile arthritis. In an aspect of the invention, the disease classification is classification of a juvenile arthritis including but not limited to, pauciarticular juvenile arthritis, polyarticular juvenile arthritis, systemic onset juvenile rheumatoid arthritis, and juvenile onset spondyloarthropathy.

In a second embodiment, the invention provides a method of determining juvenile arthritis classification in a subject exhibiting juvenile arthritis. The method involves the steps of obtaining a peripheral blood monocyte sample, a synovial fluid monocyte sample, or both, from a subject, assaying the expression level of a nucleotide sequence of interest in the samples, and comparing the expression levels of the nucleotide sequence of interest to a standard expression pattern to determine disease classification. Nucleotide sequences of interest are described elsewhere herein. In an aspect of the invention, expression levels of multiple nucleotide sequences of interest are compared to a standard expression pattern. In further aspects of the invention, expression levels of at least five, at least ten, at least fifteen, at least eighteen, or at least twenty nucleotide sequences of interest are compared to a standard expression pattern. Another aspect of the invention provides a kit for performing the method comprising a peripheral blood monocyte sample collection reagent, a synovial fluid sample collection reagent, and a detection reagent for at least one nucleotide sequence of interest. In an aspect of the invention, the kit comprises detection reagents for at least 18 nucleotide sequences of interest.

In a third embodiment, the invention provides a method of analyzing disease progression in a subject exhibiting juvenile arthritis. The method involves obtaining a first peripheral blood monocyte sample, a first synovial fluid mononuclear cell sample, or both, from the subject, assaying a first expression level of a nucleotide sequence of interest in the first samples, obtaining a second peripheral blood monocyte sample, a second synovial fluid monocyte sample, or both a second peripheral blood monocyte sample and a second synovial fluid monocyte sample, from the subject, assaying a second expression level of a nucleotide sequence of interest in the second samples, and comparing the first and second expression levels of the nucleotide sequence of interest. The invention further provides a kit for performing the method of the invention.

In a fourth embodiment, the invention provides a method of identifying a nucleotide sequence of interest expression modulating compound. The method involves obtaining a first peripheral blood monocyte sample, a first synovial fluid monocyte sample, or both from a subject exhibiting juvenile arthritis, assaying a first expression level of a nucleotide sequence of interest in the first samples, administering a compound of interest to the subject, obtaining a second peripheral blood monocyte sample, a second synovial fluid monocyte sample, or both from the subject, assaying a second expression level of a nucleotide sequence of interest in the subject, and comparing the first and second expression levels of the nucleotide sequence of interest. In an aspect of the invention, the subject is a human, mouse, rabbit, dog, pig, goat, cow, rat, monkey, chimpanzee, or sheep. In an aspect of the invention, the compound of interest is administered to a subject, cells obtained from a subject, or cells cultured from a subject.

In a fifth embodiment, the invention provides a method of identifying an arthritis modulating compound. The method involves obtaining a first peripheral blood monocyte sample and a first synovial fluid monocyte sample from a subject exhibiting juvenile arthritis, assaying a first expression level of a nucleotide sequence of interest in the first samples, administering a compound of interest to the subject, obtaining a second peripheral blood monocyte sample and a second synovial fluid monocyte sample from the subject, assaying a second expression level of a nucleotide sequence of interest in the subject, and comparing the first and second expression levels of the nucleotide sequence of interest. In an aspect of the invention, the compound of interest is administered to a subject, cells obtained from a subject, or cells cultured from a subject. The invention further provides an arthritis modulating compound identified by the method.

In a sixth embodiment, the invention provides a method of determining juvenile arthritis classification. The method involves the steps of obtaining one or more biological samples from the subject and assaying the expression pattern of nucleotide sequences of interest such as, but not limited to, CXCL chemokines in the biological samples to determine juvenile arthritis classification. In an aspect, the biological sample is a peripheral blood monocyte sample. In an aspect, the biological sample is a synovial fluid monocyte sample. In an aspect of the invention multiple biological samples, such as a peripheral blood monocyte sample and a synovial fluid monocyte sample, are obtained. Multiple biological samples may be obtained at multiple time points.

In a seventh embodiment, the invention provides a method of determining the extent of juvenile arthritis progression in a subject exhibiting juvenile arthritis. The method involves the steps of obtaining one or more biological samples from the subject and assaying the expression pattern of nucleotide sequences of interest such as, but not limited to, CXCL chemokines in the biological samples to determine juvenile arthritis classification. In an aspect, the biological sample is a peripheral blood monocyte sample. In an aspect, the biological sample is a synovial fluid monocyte sample. In an aspect of the invention multiple biological samples, such as a peripheral blood monocyte sample and a synovial fluid monocyte sample, are obtained. Multiple biological samples may be obtained at multiple time points.

In an eighth embodiment, the invention provides a method of determining disease classification in a subject. The method involves the steps of obtaining a peripheral blood monocyte sample from the subject, assaying the expression level of a nucleotide sequence of interest in the sample, and comparing the nucleotide sequence of interest expression level to a standard expression pattern to determine disease classification.

In a ninth embodiment, the invention provides a method of determining disease classification in a subject. The method involves the steps of obtaining a synovial fluid monocyte sample from the subject, assaying the expression level of a nucleotide sequence of interest in the sample, and comparing the nucleotide sequence of interest expression level to a standard expression pattern to determine disease classification.

In a tenth embodiment, the invention provides a method of determining a juvenile arthritis classification in a subject exhibiting juvenile arthritis. The method involves the steps of obtaining a peripheral blood monocyte sample from the subject, assaying the expression level of a nucleotide sequence of interest in the sample, and comparing the nucleotide sequence of interest expression level to a standard expression pattern to determine disease classification. The invention provides a kit for performing the method comprising a peripheral blood monocyte sample collection reagent and a detection reagent for a nucleotide sequence of interest.

In an eleventh embodiment, the invention provides a method of determining a juvenile arthritis classification in a subject exhibiting juvenile arthritis. The method involves the steps of obtaining a synovial fluid monocyte sample from the subject, assaying the expression level of a nucleotide sequence of interest in the sample, and comparing the nucleotide sequence of interest expression level to a standard expression pattern to determine disease classification. The invention provides a kit for performing the method comprising a synovial fluid monocyte sample collection reagent and a detection reagent for a nucleotide sequence of interest.

In a twelfth embodiment, the invention provides a method of analyzing disease progression in a subject exhibiting juvenile arthritis. The method involves the steps of obtaining a first peripheral blood monocyte sample from the subject, assaying a first expression level of a nucleotide sequence of interest in the sample, obtaining a second peripheral blood monocyte sample from the subject, assaying a second expression level of the nucleotide sequence of interest, and comparing the first and second expression levels of the nucleotide sequences of interest.

In a thirteenth embodiment, the invention provides a method of analyzing disease progression in a subject exhibiting juvenile arthritis. The method involves the steps of obtaining a first synovial fluid monocyte sample from the subject, assaying a first expression level of a nucleotide sequence of interest in the sample, obtaining a second synovial fluid monocyte sample from the subject, assaying a second expression level of the nucleotide sequence of interest, and comparing the first and second expression levels of the nucleotide sequences of interest.

In a fourteenth embodiment the invention provides a method of identifying a nucleotide sequence of interest expression modulating compound. The method involves the steps of obtaining a first peripheral blood monocyte sample from the subject, assaying a first expression level of a nucleotide sequence of interest in the sample, administering a compound of interest, obtaining a second peripheral blood monocyte sample from the subject, assaying a second expression level of the nucleotide sequence of interest, and comparing the first and second expression levels of the nucleotide sequences of interest.

In a fifteenth embodiment, the invention provides a method of identifying a nucleotide sequence of interest expression modulating compound. The method involves the steps of obtaining a first synovial fluid monocyte sample from the subject, assaying a first expression level of a nucleotide sequence of interest in the sample, administering a compound of interest, obtaining a second synovial fluid monocyte sample from the subject, assaying a second expression level of the nucleotide sequence of interest, and comparing the first and second expression levels of the nucleotide sequences of interest.

In a sixteenth embodiment, the invention provides a method of identifying an arthritis modulating compound. The method involves the steps of obtaining a first peripheral blood monocyte sample from the subject, assaying a first expression level of a nucleotide sequence of interest in the sample, administering a compound of interest, obtaining a second peripheral blood monocyte sample from the subject, assaying a second expression level of the nucleotide sequence of interest, and comparing the first and second expression levels of the nucleotide sequences of interest.

In a seventeenth embodiment, the invention provides a method of identifying an arthritis modulating compound. The method involves the steps of obtaining a first synovial fluid monocyte sample from the subject, assaying a first expression level of a nucleotide sequence of interest in the sample, administering a compound of interest, obtaining a second synovial fluid monocyte sample from the subject, assaying a second expression level of the nucleotide sequence of interest, and comparing the first and second expression levels of the nucleotide sequences of interest.

### BRIEF DESCRIPTION OF THE DRAWINGS

FIG. 1 presents expression profiles for the indicated chemokines in peripheral blood monocytes (PBMC, panels A and B) and synovial fluid monocytes (SFMC, panels C and D) obtained from juvenile arthritis patients. Expression of the indicated nucleotide sequences of interest in the tissue samples is shown relative to expression of the nucleotide sequence of interest in peripheral blood monocytes obtained from healthy individuals. Samples were obtained from patients with polyarticular juvenile arthritis (poly, hatched bars), pauciarticular juvenile arthritis (pauci, white bars), and juvenile spondyloarthropathy (JSpA, solid bars). Relative expression of ELR+ chemokines and vascular endothelial growth factor (VEGF) is presented in panels A and C. Relative expression of ELR− chemokines is presented in panels B and D. Asterisks indicate a p<0.05 relative to pauciarticular samples when analyzed by the students t test.

FIG. 2 presents relative expression values of various nucleotide sequences of interest in patients exhibiting a pauciarticular course, a polyarticular course, and in juvenile spondyloarthropathy (JSpA).

### DETAILED DESCRIPTION OF THE INVENTION

The present invention provides methods of classifying disease, particularly juvenile arthritides, determining disease progression, predicting disease course, and identifying anti-arthritic compounds. Compositions of the invention include kits for performing the methods of the invention and anti-arthritic compounds identified by a method of the invention. The invention relates to differential expression of nucleotide sequences of interest in peripheral blood monocytes (PBMCs) and synovial fluid monocytes (SFMCs) in the various types of juvenile arthritis.

By “disease classification,” “disease type,” or “disease subtype” is intended a set of diseases limited by certain shared characteristics, phenotypes, genotypes, or traits. Disease classifications for various disease and disorders are known in the art. As discussed elsewhere herein, multiple disease classification systems exist for juvenile arthritis. Juvenile arthritis classification systems include, but are not limited to, criteria developed by the ACR, EULAR, and the International League of Associations for Rheumatology. Classification criteria among these three systems vary, but in all three the subtypes of juvenile arthritis are generally characterized by the number of affected joints within six months of onset. Juvenile arthritis classifications include, but are not limited to, pauciarticular arthritis, polyarticular arthritis, systemic onset juvenile rheumatoid arthritis, and juvenile onset spondyloarthropathy. Disease course or disease progression is often described in terms of the number of affected joints after six months of age and include, but are not limited to, a pauciarticular course and a polyarticular course.

By “subject” is intended a mammal, e.g., a human, or an experimental or animal or disease model or mammalian tissue or mammalian cells. Suitable subjects include mammals, particularly humans, exhibiting a juvenile arthritis, tissue obtained from a mammal exhibiting a juvenile arthritis, cells obtained from a mammal exhibiting a juvenile arthritis, and cells cultured from a mammal exhibiting a juvenile arthritis. The subject can also be a non-human animal such as, but not limited to, a horse, hamster, guinea pig, mouse, rabbit, dog, pig, goat, cow, rat, monkey, chimpanzee, sheep, or other domestic animal.

By “biological sample” is intended a sample collected from a subject including, but not limited to, tissues, cells, mucosa, fluid, scrapings, hairs, cell lysates, and secretions, particularly peripheral blood monocytes and synovial fluid monocytes. A peripheral blood monocyte sample comprises at least one monocyte cell obtained from peripheral blood. A synovial fluid monocyte sample comprises at least one monocyte obtained from synovial fluid. Biological samples such as peripheral blood monocyte samples and synovial fluid monocyte samples can be obtained by any method known to one skilled in the art. Further, biological samples such as, but not limited to peripheral blood monocytes and synovial fluid monocyte samples can be enriched, purified, or isolated by any method known to one skilled in the art.

An “isolated” or “purified” biological sample, particularly a peripheral blood monocyte sample or synovial fluid monocyte sample is substantially or essentially free from components that normally accompany or interact with the sample as found in its naturally occurring environment. Thus, an isolated or purified PBMC or SFMC sample is substantially free of other cell types. A PBMC or SFMC sample that is substantially free of extraneous material includes preparations of monocytes having less than about 30%, 20%, 10%, 5%, or 1% (by dry weight) of contaminating cells. Methods of isolating peripheral blood monocytes from whole blood are known in the art and include, but are not limited to, Ficoll gradient centrifugation and ultracentrifugation (de Jager et al. (2003) Clin. & Diag. Lab. Immun. 10: 133-139, herein incorporated by reference in its entirety). Methods of isolating synovial fluid mononuclear cells are known in the art and include, but are not limited to, Ficoll gradient centrifugation of synovial fluid. In an aspect of the invention, the process of isolating monocytes from a source sample begins within about 24 hours of collection, preferably within about 12 hours of collection, more preferably within about 8 hours of collection, yet more preferably within about 4 hours of collection of the source sample.

Methods of assaying expression levels are known in the art and include, but are not limited to, qualitative Western blot analysis, immunoprecipitation, radiological assays, polypeptide purification, spectrophotometric analysis, Coomassie staining of acrylamide gels, ELISAs, RT-PCR, 2-D gel electrophoresis, microarray analysis, in situ hybridization, chemiluminescence, silver staining, enzymatic assays, ponceau S staining, multiplex RT-PCR, immunohistochemical assays, radioimmunoassay, colorimetric analysis, immunoradiometric assays, positron emission tomography, Northern blotting, fluorometric assays, fluorescence activated cell sorter staining of permeabilized cells, radioimmunosorbent assays, real-time PCR, hybridization assays, sandwich immunoassays, flow cytometry, SAGE, differential amplification, or electronic analysis. See, for example, Ausubel et al, eds. (2002) Current Protocols in Molecular Biology, Wiley-Interscience, New York, N.Y.; Coligan et al (2002) Current Protocols in Protein Science, Wiley-Interscience, New York, N.Y.; Sun et al. (2001) Gene Ther. 8: 1572-1579; de Jager et al. (2003). Clin. & Diag. Lab. Immun. 10: 133-139; U.S. Pat. Nos. 6,489,4555; 6,551,784; 6,607,879; 4,981,783; and 5,569,584; herein incorporated by reference in their entirety.

Nucleotide sequences of interest in the present invention include numerous inflammatory related compounds. Nucleotide sequences of interest in the present invention include, but are not limited to, CXCL1 (SEQ ID NO:1), CXCL2 (SEQ ID NO:3), CXCL3 (SEQ ID NO:5), CXCL4 (SEQ ID NO:7), CXCL5 (SEQ ID NO:9), CXCL7 (SEQ ID NO:1), CXCL8 (SEQ ID NO:13), CXCL9 (SEQ ID NO:15), CXCL10 (SEQ ID NO:17), CXCL11 (SEQ ID NO:19), CXCL13 (SEQ ID NO:21), VEGF (SEQ ID NO:55), CCL2 (SEQ ID NO:25), AREG (SEQ ID NO:35), PBEF (SEQ ID NO:23), PHLDA (SEQ ID NO:27), SERPINB2 (SEQ ID NO:29), TGIF (SEQ ID NO:31), and THBD (SEQ ID NO:33).

These compounds include, but are not limited to, cytokines and chemokines. A cytokine is a general term for a mediator released primarily but not exclusively by a cell population of the immune system as a response to a specific stimulating agent, e.g., a specific antigen or an alloantigen; or a non-specific, polyclonal activator, e.g. an endotoxin or other cell wall components. Chemokines are members of the large superfamily of inducible small cytokines and can be divided into at least four groups according to a conserved structural motif of the first two closely paired cysteines within their amino acid sequence. The CXC chemokines have a single amino acid separating the first two cysteines. CXCL chemokines represent specific ligands of the CXCRs (Cys-X-Cys receptor). Of particular interest is the family of CXCL chemokines including, but not limited to, CXCL1 (SEQ ID NO:1), CXCL2 (SEQ ID NO:3), CXCL3 (SEQ ID NO:5), CXCL4 (SEQ ID NO:7), CXCL5 (SEQ ID NO:9), CXCL7 (SEQ ID NO:11), CXCL8 (SEQ ID NO:13), CXCL9 (SEQ ID NO:15), CXCL10 (SEQ ID NO:17), CXCL11 (SEQ ID NO:19), and CXCL13 (SEQ ID NO:21), and VEGF (Genbank NM003376; SEQ ID NO:55). As used herein, a nucleotide sequence of interest is any nucleotide sequence having a nucleotide sequence set forth in SEQ ID NOS:1, 3, 5, 7, 9, 11, 13, 15, 17, 19, 21, 23, 25, 27, 29, 31, 33, 35, 37, 38, 39, 40, 41, 42, 43, 44, 45, 46, 47, 48, 49, 50, 51, 52, 53, 54, and 55; a nucleotide sequence having at least 90% identity to a nucleotide sequence set forth in SEQ ID NOS: 1, 3, 5, 7, 9, 11, 13, 15, 17, 19, 21, 23, 25, 27, 29, 31, 33, 35, 37, 38, 39, 40, 41, 42, 43, 44, 45, 46, 47, 48, 49, 50, 51, 52, 53, 54, and 55; a nucleotide sequence having an amino acid sequence set forth in SEQ ID NOS: 2, 4, 6, 8, 10, 12, 14, 16, 18, 20, 22, 24, 26, 28, 30, 32, 34, 36, and 56; a nucleotide sequence having an amino acid sequence having at least 90% identity to an amino acid sequence set forth in SEQ ID NOS:2, 4, 6, 8, 10, 12, 14, 16, 18, 20, 22, 24, 26, 28, 30, 32, 34, 36, and 56; or fragment thereof.

Fragments and variants of the nucleotide sequences of interest and proteins encoded thereby are also encompassed by the present invention. By “fragment” is intended a portion of the nucleotide sequence or a portion of the amino acid sequence and hence protein encoded thereby. Fragments of a nucleotide sequence may encode protein fragments that retain the biological activity of the native protein and hence exhibit activity Alternatively, fragments of a nucleotide sequence that are useful as hybridization probes generally do not encode fragment proteins retaining biological activity. Thus, fragments of a nucleotide sequence may range from at least about 20 nucleotides, about 50 nucleotides, about 100 nucleotides, and up to the full-length nucleotide sequence encoding the proteins of the invention.

A fragment of a nucleotide sequence of interest that encodes a biologically active portion of a nucleotide sequence of interest will encode at least 15, 25, 30, 50, 100, 150, 200, 250, 300, 350, 400, 415, or up to the total number of amino acids present in a full-length nucleotide sequence of interest. Fragments of a nucleotide sequence of interest that are useful as hybridization probes or PCR primers generally need not encode a biologically active portion of a polypeptide encoded by a nucleotide sequence of interest.

Thus, a fragment of a nucleotide sequence of interest may encode a biologically active portion of a nucleotide sequence of interest or it may be a fragment that can be used as a hybridization probe or PCR primer using methods disclosed below. A biologically active portion of a nucleotide sequence of interest can be prepared by isolating a portion of one of the nucleotide sequences of the invention, expressing the encoded portion of the protein (e.g., by recombinant expression in vitro), and assessing the activity of the encoded portion of the protein. Nucleic acid molecules that are fragments of a nucleotide sequence of interest comprise at least 16, 20, 50, 75, 100, 150, 200, 250, 300, 350, 400, 450, 500, 550, 600, 650, 700, 800, 900, 1,000, 1,100, 1,200, 1,300, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 2950, 3000, 3050, 3100, 3150, 3200, 3250, 3300, 3350, 3400, 3450, 3500, 3550, 3600, 3650, 3700, 3750, 3800, 3850, 3900, 4000, 4050, 4100, 4150, 4200, 4212 nucleotides, or up to the number of nucleotides present in a full-length nucleotide sequence of interest disclosed herein. Fragments of interest include, but are not limited to, the nucleotide sequences set forth in SEQ ID NOS:37-54.

By “variants” is intended substantially similar sequences. For nucleotide sequences, conservative variants include those sequences that, because of the degeneracy of the genetic code, encode the amino acid sequence of one of the nucleotide sequence of interest polypeptides. Naturally occurring allelic variants such as these can be identified with the use of well-known molecular biology techniques, as, for example, with polymerase chain reaction (PCR) and hybridization techniques as outlined below. Variant nucleotide sequences also include synthetically derived nucleotide sequences, such as those generated, for example, by using site-directed mutagenesis but which still encode a nucleotide sequence of interest. Generally, variants of a particular nucleotide sequence of the invention will have at least about 90%, 91%, 92%, 93%, 94%, 95%, 96%, 97%, and more preferably at least about 98%, 99% or more sequence identity to that particular nucleotide sequence as determined by sequence alignment programs described elsewhere herein using default parameters.

By “variant” protein is intended a protein derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Variant proteins encompassed by the present invention are biologically active, that is they continue to possess the desired biological activity of the native protein. Such variants may result from, for example, genetic polymorphism or from human manipulation. Biologically active variants of a native polypeptide encoded by a nucleotide sequence of interest will have at least about 90%, 91%, 92%, 93%, 94%, 95%, 96%, 97%, and more preferably at least about 98%, 99% or more sequence identity to the amino acid sequence for the native protein as determined by sequence alignment programs described elsewhere herein using default parameters. A biologically active variant of a protein of the invention may differ from that protein by as few as 1-15 amino acid residues, as few as 1-10, such as 6-10, as few as 5, as few as 4, 3, 2, or even 1 amino acid residue.

The proteins of the invention may be altered in various ways including amino acid substitutions, deletions, truncations, and insertions. Methods for such manipulations are generally known in the art. For example, amino acid sequence variants of the polypeptides encoded by the nucleotide sequences of interest can be prepared by mutations in the DNA. Methods for mutagenesis and nucleotide sequence alterations are well known in the art. See, for example, Kunkel (1985) Proc. Natl. Acad. Sci. USA 82: 488-492; Kunkel et al. (1987) Methods in Enzymol. 154: 367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be preferable.

Thus, the genes and nucleotide sequences of the invention include both the naturally occurring sequences as well as mutant forms. Likewise, the proteins of the invention encompass both naturally occurring proteins as well as variations and modified forms thereof. Such variants will continue to possess the desired expression pattern. Obviously, the mutations that will be made in the DNA encoding the variant must not place the sequence out of reading frame and preferably will not create complementary regions that could produce secondary mRNA structure. See, EP Patent Application Publication No. 75,444.

The nucleotide sequences disclosed herein can be used to identify corresponding sequences in cells, tissues, and animals. In this manner, methods such as PCR, hybridization, microarrays, and the like can be used to assay expression of such sequences based on their sequence homology to the sequences set forth herein. These techniques may be used as a diagnostic assay to determine expression levels of the nucleotide sequences of interest in an animal or animal cell.

In a PCR approach, oligonucleotide primers can be designed for use in PCR reactions to amplify corresponding DNA sequences from cDNA or genomic DNA extracted from any animal of interest. Methods for designing PCR primers and PCR cloning are generally known in the art and are disclosed in Sambrook et al. (1989) Molecular Cloning: A Laboratory Manual (2d ed., Cold Spring Harbor Laboratory Press, Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers, and the like.

In hybridization techniques, all or part of a known nucleotide sequence is used as a probe that selectively hybridizes to other corresponding nucleotide sequences present in a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides, and may be labeled with a detectable group such as ³²P, or any other detectable marker. Thus, for example, probes for hybridization can be made by labeling synthetic oligonucleotides based on the nucleotide sequences of interest. Methods for preparation of probes for hybridization and for construction of cDNA and genomic libraries are generally known in the art and are disclosed in Sambrook et al. (1989) Molecular Cloning: A Laboratory Manual (2d ed., Cold Spring Harbor Laboratory Press, Plainview, N.Y.).

For example, an entire nucleotide sequence of interest disclosed herein, or one or more portions thereof, may be used as a probe capable of specifically hybridizing to corresponding nucleotide sequences of interest. To achieve specific hybridization under a variety of conditions, such probes include sequences that are unique among nucleotide sequences of interest and are preferably at least about 10 nucleotides in length, and most preferably at least about 20 nucleotides in length. Such probes may be used to amplify corresponding nucleotide sequence of interest from a chosen subject by PCR.

Hybridization techniques include hybridization screening of plated DNA libraries (either plaques or colonies; see, for example, Sambrook et al. (1989) Molecular Cloning: A Laboratory Manual (2d ed., Cold Spring Harbor Laboratory Press, Plainview, N.Y.).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, preferably less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C. Duration of hybridization is generally less than about 24 hours, usually about 4 to about 12 hours.

Specificity is typically the function of post-hybridization washes, the critical factors being the ionic strength and temperature of the final wash solution. For DNA—DNA hybrids, the Tₘ can be approximated from the equation of Meinkoth and Wahl (1984) Anal. Biochem. 138: 267-284: Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization, and/or wash conditions can be adjusted to hybridize to sequences of the desired identity. For example, if sequences with approximately 90% identity are sought, the Tₘ can be decreased 110° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 110° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), it is preferred to increase the SSC concentration so that a higher temperature can be used. An extensive guide to the hybridization of nucleic acids is found in Tijssen (1993) Laboratory Techniques in Biochemistry and Molecular Biology—Hybridization with Nucleic Acid Probes, Part I, Chapter 2 (Elsevier, N.Y.); and Ausubel et al., eds. (1995) Current Protocols in Molecular Biology, Chapter 2 (Greene Publishing and Wiley-Interscience, New York). See Sambrook et al. (1989) Molecular Cloning: A Laboratory Manual (2d ed., Cold Spring Harbor Laboratory Press, Plainview, N.Y.). Thus, isolated sequences that have inflammatory related activity and which hybridize under stringent conditions to the nucleotide sequences of interest disclosed herein, or to fragments thereof, are encompassed by the present invention. Such sequences will be at least 90%, 91%, 92%, 93%, 94%, 95%, 96%, 97%, 98% to 99% homologous or more with the disclosed sequences. That is, the sequence identity of sequences may range, sharing at least 90%, 91%, 92%, 93%, 94%, 95%, 96%, 97%, 98%, 99% or more sequence identity.

The following terms are used to describe the sequence relationships between two or more nucleic acids or polynucleotides: (a) “reference sequence”, (b) “comparison window”, (c) “sequence identity”, (d) “percentage of sequence identity”, and (e) “substantial identity”.

(a) As used herein, “reference sequence” is a defined sequence used as a basis for sequence comparison. A reference sequence may be a subset or the entirety of a specified sequence; for example, as a segment of a full-length cDNA or gene sequence or the complete cDNA or gene sequence.

(b) As used herein “comparison window” makes reference to a contiguous and specified segment of a polynucleotide sequence, wherein the polynucleotide sequence in the comparison window may comprise additions or deletions (i.e. gaps) compared to the reference sequence (which does not comprise additions or deletions) for optimal alignment of the two sequences. Generally the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent sequence identity between any two sequences can be accomplished using a mathematical algorithm. Preferred, non-limiting examples of such mathematical algorithms are the algorithm of Myers and Miller (1988) CABIOS 4: 11-17; the local homology algorithm of Smith et al. (1981) Adv. Appl. Math. 2: 482; the homology alignment algorithm of Needleman and Wunsch (1970) J. Mol. Biol. 48: 443-453; the search-for-similarity-method of Pearson and Lipman (1988) Proc. Natl. Acad. Sci. 85: 2444-2448; the algorithm of Karlin and Altschul (1990) Proc. Natl. Acad. Sci. USA 87: 2264, modified as in Karlin and Altschul (1993) Proc. Natl. Acad. Sci. USA 90: 5873-5877.

Computer implementations of these mathematical algorithms can be utilized for comparison of sequences to determine sequence identity. For purposes of the present invention, comparison of nucleotide or protein sequences for determination of percent sequence identity to the sequences disclosed herein is preferably made using the GCG program GAP (Version 10.00 or later) with its default parameters or any equivalent program. By “equivalent program” is intended any sequence comparison program that, for any two sequences in question, generates an alignment having identical nucleotide or amino acid residue matches and an identical percent sequence identity when compared to the corresponding alignment generated by the preferred program.

Sequence comparison programs include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters. The CLUSTAL program is well described by Higgins et al. (1988) Gene 73: 237-244 (1988); Higgins et al. (1989) CABIOS 5: 151-153; Corpet et al. (1988) Nucleic Acids Res. 16: 10881-90; Huang et al. (1992) CABIOS 8: 155-65; and Pearson et al. (1994) Meth. Mol. Biol. 24: 307-331. The ALIGN program is based on the algorithm of Myers and Miller (1988) supra. A PAM 120 weight residue table, a gap length penalty of 12, and a gap penalty of 4 can be used with the ALIGN program when comparing amino acid sequences. The BLAST programs of Altschul et al. (1990) J. Mol. Biol. 215: 403 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. To obtain gapped alignments for comparison purposes, Gapped BLAST (in BLAST 2.0) can be utilized as described in Altschul et al. (1997) Nucleic Acids Res. 25: 3389. Alternatively, PSI-BLAST (in BLAST 2.0) can be used to perform an iterated search that detects distant relationships between molecules. See Altschul et al. (1997) supra. When utilizing BLAST, Gapped BLAST, PSI-BLAST, the default parameters of the respective programs (e.g., BLASTN for nucleotide sequences, BLASTX for proteins) can be used. See http://www.ncbi.nlm.nih.gov. Alignment may also be performed manually by inspection.

(c) As used herein, “sequence identity” or “identity” in the context of two nucleic acid or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” of polynucleotide sequences means that a polynucleotide comprises a sequence that has at least 70% sequence identity, preferably at least 80%, more preferably at least 90%, and most preferably at least 95%, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 60%, more preferably at least 70%, 80%, 90%, and most preferably at least 95%.

Another indication that nucleotide sequences are substantially identical is if two molecules hybridize to each other under stringent conditions. Generally, stringent conditions are selected to be about 5° C. lower than the thermal melting point (Tₘ) for the specific sequence at a defined ionic strength and pH. However, stringent conditions encompass temperatures in the range of about 1° C. to about 20° C. lower than the Tₘ, depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

(e)(ii) The term “substantial identity” in the context of a peptide indicates that a peptide comprises a sequence with at least 70% sequence identity to a reference sequence, preferably 80%, more preferably 85%, most preferably at least 90% or 95% sequence identity to the reference sequence over a specified comparison window. Preferably, optimal alignment is conducted using the homology alignment algorithm of Needleman and Wunsch (1970) J. Mol. Biol. 48: 443-453. An indication that two peptide sequences are substantially identical is that one peptide is immunologically reactive with antibodies raised against the second peptide. Thus, a peptide is substantially identical to a second peptide, for example, where the two peptides differ only by a conservative substitution. Peptides that are “substantially similar” share sequences as noted above except that residue positions that are not identical may differ by conservative amino acid changes.

Expression of the nucleotide sequences of interest in the present invention differs between cell types and among classifications of juvenile arthritis. Expression differences include, but are not limited to, the following variations in expression. Expression of CXCL1, CXCL2, CXCL3, and CXCL8 in peripheral blood monocytes (PBMCs) from polyarticular subjects was higher than in PBMCs from pauciarticular subjects and healthy controls. However, expression levels of these chemokines were equivalent in synovial fluid monocyte (SFMCs) samples from patients with the various disease classifications. Expression of CXCL9, CXCL10, and CXCL11 was lower in SFMCs from polyarticular patients compared with SFMCs from pauciarticular patients. Expression of CXCL4 and CXCL10 in SFMCs from juvenile onset spondyloarthropathies was lower than expression in SFMCs from pauciarticular patients. Expression of the angiostatic chemokines in PBMCs was essentially the same between juvenile arthritis classifications. Thus the methods of the invention are based on the differential expression of one or more nucleotide sequences of interest in one or more cell types.

“Differential expression” as used herein refers to both quantitative as well as qualitative differences in the genes' temporal and/or tissue expression patterns. Thus, a differentially expressed nucleotide sequence of interest may have its expression activated or completely inactivated among the disease classifications or under control versus experimental conditions. Such a qualitatively regulated gene will exhibit an expression pattern within a given tissue or cell type which is detectable in either healthy subjects or subjects with a juvenile arthritides. The expression of a nucleotide sequence of interest is detectable in 0, 1, 2, 3, 4 or more classifications of juvenile arthritis. Alternatively, such a qualitatively regulated gene will exhibit an expression pattern within a given tissue or cell type which is detectable in either control or experimental subjects, but is not detectable in both. Alternatively, a differentially expressed gene may have its expression modulated, i.e., quantitatively increased or decreased, among the juvenile arthritis subtypes, in normal versus disease states, or under control versus experimental conditions. Transcript levels of differentially expressed genes may vary by 0.01%, 0.1%, 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 100% or more.

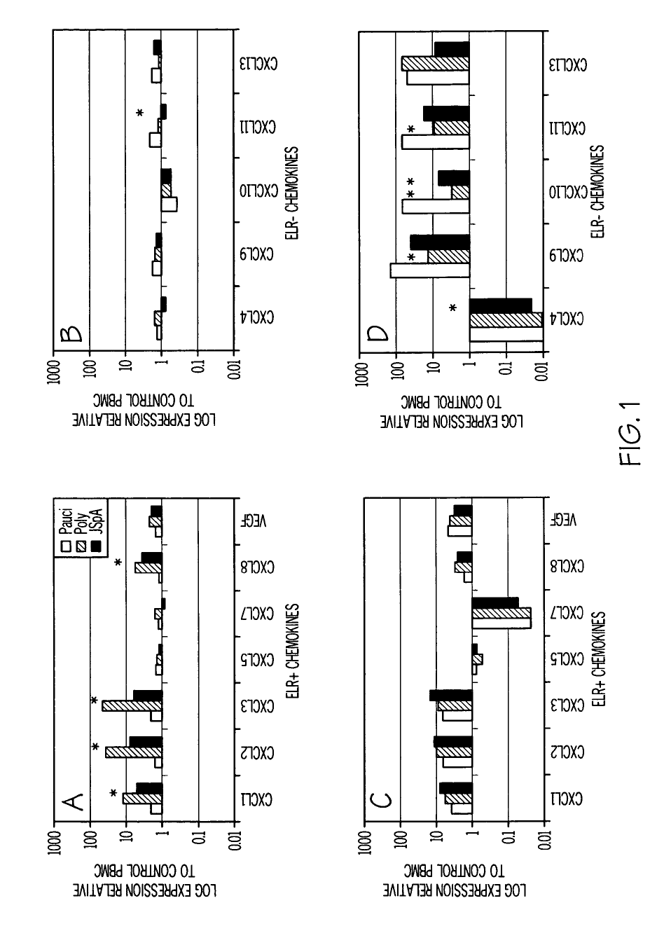

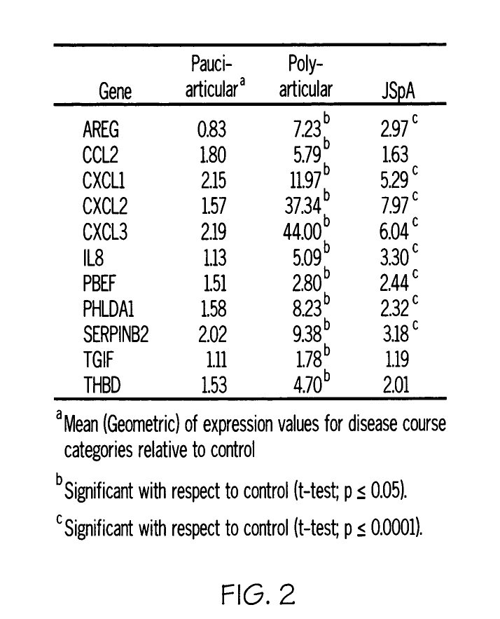

By “expression pattern” is intended a description of the relative expression levels of one or more nucleotide sequences in one or more cell types. The expression pattern of 1, 2, 3, 4, 5, 6, 7, 8, 9, 10, 11, 12, 13, 14, 15, 16, 17, or 18 nucleotide sequences of interest is used in the methods of the invention. A standard expression pattern is a predetermined description of the relative expression levels of one or more nucleotide sequences in one or more cell types. For example, a standard expression pattern might describe the relative expression levels of one or more nucleotide sequences of interest in peripheral blood monocytes, in synovial fluid monocytes, or in both peripheral blood monocytes and synovial fluid monocytes. Expression patterns of the nucleotide sequences of interest in PBMCs and SFMCs differ among the various classifications of juvenile arthritis. The expression patterns indicated in FIG. 1 and FIG. 2 are examples of standard expression patterns suitable for determining the classification of juvenile arthritis that a patient exhibits. Expression levels of one or more nucleotide sequences of interest in samples obtained from a subject are compared to standard expression patterns indicative of juvenile arthritis classification.

By “disease progression” or “disease course” is intended the physiological events related to a disease or disorder that occur during the period after the initial presentation in a subject or patient exhibiting a disease or disorder. With respect to juvenile arthritis, the number of affected joints beyond the first six months of disease is used to describe the disease course or progression. A pauciarticular course involves four or fewer joints; a polyarticular course involves five or more joints. Systemic onset juvenile rheumatoid arthritis (SOJRA) often presents pauciarticularly but typically progresses polyarticularly. Juvenile onset spondyloarthropathy (JSpA) often presents pauciarticularly, and many patients with JSpA do not progress polyarticularly. Identifying the disease course or disease progression that an untreated patient would experience allows practitioners to identity those patients who would benefit most from aggressive early intervention. Identifying compounds or agents that alter disease progression is an embodiment of the invention. Expression profiles of the nucleotide sequences of interest may be used to analyze disease progression or disease course in a subject with juvenile arthritis.

Juvenile arthritis phenotypes include, but are not limited to, expression of the nucleotide sequences of interest, number of affected joints, apoptotic tendency, erythrocyte sedimentation rate, C reactive protein levels, IL15 expression levels, joint pain, joint swelling, joint stiffness, irritability, iritis, iridocyclitis, uveitis, fevers, rashes, rheumatoid factor presence, synovial thickening, synovial joint space expansion, effusion in the suprapatellar pouch, bone mineral content, bone mineral density, (See Petty, R. E. and Cassidy, J. T. (2001) The Juvenile Idiopathic Arthritides. In Textbook of Pediatric Rheumatology. 4^{th} ed. J. T. Cassidy and R. E. Petty, ed. W.B. Sauders Co., St. Louis; Cassidy et al. (1986) Arthritis Rheum. 29: 274, Petty et al. (1998) J. Rheumatol. 25: 1991; and Smolewska et al. (2003) Ann. Rheum. Dis. 62: 761-763; herein incorporated by reference in their entirety.

Methods of measuring juvenile arthritis phenotypes are known in the art and include, but are not limited to, methods of assaying expression described elsewhere herein, TUNEL, arthrosonography, and dual energy x-ray absorptiometry. See Bendtzen et al. (2003) Clin. Exp. Immunol. 134: 151-158; Smolewska et al. (2003) Ann. Rheum. Dis. 62: 761-763; Lien et al. (2003) Arthritis Rheum. 48: 2214-2223, the Childhood Health Assessment Questionnaire, Textbook of pediatric Rheumatology. 4^{th} ed. J. T. Cassidy and R. E. Petty, ed. W.B. Sauders Co., St. Louis; Cassidy et al. (1986) Arthritis Rheum. 29: 274, Petty et al. (1998) J. Rheumatol. 25: 1991; and Smolewska et al. (2003) Ann. Rheum. Dis. 62: 761-763; herein incorporated by reference in their entirety.

In an embodiment, expression levels of nucleotide sequences of interest may be used to identify nucleotide sequence of interest expression modulating compounds. A “nucleotide sequence of interest expression modulating compound” is a compound that modulates expression of a nucleotide sequence of interest. Modulation may be an increase or decrease in expression of the nucleotide sequence of interest in one or more samples from a subject. A nucleotide sequence of interest expression modulating compound will modulate expression of a nucleotide sequence of interest by at least 1%, 5%, preferably 10%, 20%, more preferably 30%, 40%, 50%, 60%, yet more preferably 70%, 80%, 90%, or 100% as compared to an untreated or placebo treatment effect. Modulation of expression of a nucleotide sequence of interest may occur in only one tissue or it may occur in multiple tissues. Methods for assaying expression of nucleotide sequences of interest are described elsewhere herein. Any method of assaying expression of a nucleotide sequence of interest known in the art may be used to monitor the effects of the compound of interest on a subject.

To identify nucleotide sequence of interest expression modulating compounds, a first biological sample is obtained from a subject, particularly a subject exhibiting juvenile arthritis. A first biological sample is a first peripheral blood monocyte sample, a first synovial fluid monocyte sample, or both a first peripheral blood monocyte sample and a first synovial fluid monocyte sample. A compound of interest is administered to the subject. After administration of either the compound of interest or a placebo, the subject is incubated for a period of time. The period of time will have a predetermined duration appropriate to analysis of a juvenile arthritis phenotype. Such durations include, but are not limited to, 30 seconds; 1, 5, 10, 30, or 60 minutes; 8, 12, 24, 36, or 48 hours; 3,4,5,6, or 7 days; 2,3, or 4 weeks; 2, 3, 4, 5, 6, 7, 8, 9, 10, 11, or 12 months; up to 3 years. A second biological sample is obtained from the subject. A second biological sample is a second peripheral blood monocyte sample, a second synovial fluid monocyte sample, or both a second peripheral blood monocyte sample and a second synovial fluid monocyte sample. Monitoring of expression of a nucleotide sequence of interest may occur continuously; at a single interval; or at multiple intervals, such as, but not limited to, hourly, daily, weekly, and monthly. Any method of assaying expression of a nucleotide sequence of interest known in the art may be used to monitor the effects of the compound of interest on a transgenic animal of the invention.

In an embodiment, the nucleotide sequences of interest in the invention may be used to identify an arthritis modulating compound. An “arthritis modulating compound” is a compound that modulates an arthritic phenotype. Modulation may be an increase or decrease in an arthritic phenotype. An arthritis modulating compound will modulate a arthritic phenotype by at least 1%, 5%, preferably 10%, 20%, more preferably 30%, 40%, 50%, 60%, yet more preferably 70%, 80%, 90%, or 100% as compared to an untreated or placebo treatment effect. Methods for assaying arthritic phenotypes are described elsewhere herein. Any method of assaying an arthritic phenotype known in the art may be used to monitor the effects of the compound of interest on the subject.

To identify arthritis modulating compounds, a first peripheral blood monocyte sample and a first synovial fluid monocyte sample are obtained from a subject exhibiting juvenile arthritis or cells or tissues from a mammal exhibiting juvenile arthritis. A compound of interest is administered to the subject. After administration of either the compound of interest or a placebo, the subject is incubated for a period of time. The period of time will have a predetermined duration appropriate to analysis of the phenotype. Such durations include, but are not limited to, 30 seconds; 1, 5, 10, 30, or 60 minutes; 8, 12, 24, 36, or 48 hours; 3, 4, 5, 6, or 7 days; 2, 3, or 4 weeks; 2, 3, 4, 5, 6, 7, 8, 9, 10, 11, or 12 months; up to 3 years. A second peripheral blood monocyte sample and/or a second synovial fluid monocyte sample is obtained. Monitoring of an arthritis phenotype may occur continuously; at a single interval; or at multiple intervals, such as, but not limited to, hourly, daily, weekly, and monthly. Any method of assaying an arthritis phenotype known in the art may be used to monitor the effects of the compound of interest on the subject.

The term “administer” is used in its broadest sense and includes any method of introducing a compound into a subject such as, but not limited to, a human, mouse, rabbit, dog, pig, goat, cow, rat, monkey, chimpanzee, or sheep. This includes producing polypeptides or polynucleotides in vivo as by transcription or translation in vivo of polynucleotides that have been exogenously introduced into a subject. Thus, polypeptides or nucleic acids produced in the subject from the exogenous compositions are encompassed in the term “administer.”

A “compound” comprises, but is not limited to, nucleic acid molecules, aldosterone antagonists, polypeptides, peptides, peptidomimetics, glycoproteins, transcription factors, small molecules, chemokine receptors, antisense nucleotide sequences, chemokine receptor ligands, lipids, antibodies, receptor inhibitors, ligands, sterols, steroids, hormones, chemokine receptor agonists, chemokine receptor antagonists, agonists, antagonists, ion-channel modulators, diuretics, enzymes, enzyme inhibitors, carbohydrates, deaminases, deaminase inhibitors, G-proteins, G-protein receptor inhibitors, ACE inhibitors, hormone receptor modulators, alcohols, reverse transcriptase inhibitors, neurotransmitter inhibitors, neurotransmitter receptor modulators, hormones, phosphatases, lactones, and vasodilators. A compound may additionally comprise a pharmaceutically acceptable carrier.

As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, such media can be used in the compositions of the invention. Supplementary active compounds can also be incorporated into the compositions. A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a carboxypeptidase protein or anti-carboxypeptidase antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For oral administration, the agent can be contained in enteric forms to survive the stomach or further coated or mixed to be released in a particular region of the GI tract by known methods. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. “Dosage unit form” as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Arthritis modulating compounds identified by the methods of this invention may be used in the treatment of human individuals.

In an embodiment, the invention provides kits for performing the methods of the invention. Such kits comprise a collection reagent and a detection reagent. By “collection reagent” is intended any substance that facilitates collection of the indicated substance. Collection reagents that facilitate the reaction may or may not participate in the purification or enrichment of the desired cell type in the sample. Collection reagents include, but are not limited to, syringes, needles, tubing, butterfly syringes, plastic vials, glass vials, centrifuges, Ficoll, ultracentrifuges, vessels, such as microfuge tubes and multiwell plates; measuring devices, such as micropipette tips and capillary tubes; filters; separation devices such as microfuge tube filter inserts, vacuum apparati, purification resins, magnetic beads, and columns; reagents; compounds; solutions; molecules; buffers; inhibitors; chelating agents; ions; terminators; stabilizers; precipitants; solubilizers; acids; bases; salts; reducing agents; oxidizing agents; enzymes; catalysts; and denaturants.

By “detection reagent” is intended any substance that facilitates detection of the expression of a nucleotide sequence of interest. Detection reagents include, but are not limited to, microchips, microarrays, primers, probes, antibodies, nucleic acid probes, fluorescently labeled primers, fluorescently labeled antibodies, radiolabeled antibodies, radiolabeled primers, radiolabeled probes, expression analyzing reagents, and other reagents known to one of skill in the art. In an embodiment of the invention, a concentrated detection reagent is provided in kits of the invention. The concentration of a detection reagent provided in a kit of the invention may be 1, 2, 3, 4, 5, 6, 7, 8, 9, 10, 15, 20, 25, 30, 35, 40, 50, 60, 70, 80, 90, 100, or more fold concentrated than the desired concentration of the detection reagent in a detection reaction. In an embodiment a kit provides a detection reagent and a transfer component. A “transfer component” is a material that facilitates transfer of the detection reagent to a processing facility. Transfer components include but are not limited to, packages, boxes, shipping labels, and envelopes. Use of a processing facility to process the detection reagent enhances instrumentation and protocol standardization which may be of particular benefit when the detection reagent is a microchip or microarray.

The following examples are offered by way of illustration and not limitation.

### EXPERIMENTAL

### Example 1

### Collection of Juvenile Arthritis Cell Samples

Twenty-seven chronic juvenile arthritis patients were classified according to disease course: pauciarticular (n=5; age 12.9±3.9 years) polyarticular (n=15; age 15.9±±4.9 years) systemic (n=1; age 13.8 years) course or JSpA (n=6, age 21.2±4.9 years). Whole blood was collected in EDTA from the patients during scheduled clinical visits during a period of active disease. During the same visit matched synovial fluid samples were obtained from 21 patients (10 polyarticular, 5 pauciarticular, 5 JSpA, and 1 systemic). Peripheral blood was obtained from 11 healthy controls. Cells were isolated by Ficoll gradient centrifugation and frozen in 90% fetal calf serum/10% DMSO at 8-10×10⁶ cells/ml prior to RNA isolation with Trizol (Invitrogen Life Technologies; Carlsbad, Calif.). RNA was isolated according to the manufacturer's recommended protocol.

### Example 2

### Expression Profiling of Peripheral Blood Monocyte Samples and Synovial Fluid Monocytes

Biotinylated cRNA was synthesized from total RNA using manufacturer's recommended protocols (Enzo; Farmingdale N.Y.). The labeled cRNA was processed as recommended by the Affymetrix GeneChip Expression Analysis Technical Manual (Affymetrix; Santa Clara, Calif.). Labeled cRNA was hybridized to Affymetrix U95A chips. Quality was assessed and expression values were derived using Microarray Suite 5.0 (MAS 5.0; Affymetrix).

### Example 3

### Real-Time RT-PCR Analysis of CXCL3 and CXCL8

Real-time RT-PCR was performed on total RNA prepared separately from that used in the microarray expression analysis. The real-time RT-PCR was performed using Assays-on-Demand reagents from Applied Biosystems, Foster City Calif. and using the manufacturer's recommended protocols. Real time RT-PCR was used to amplify CXCL3 (Hs00171061_ml), CXCL8 (Hs00174103_ml), and GAPDH (Hs00174103_ml) from total RNA.

All publications, patents, and patent applications mentioned in the specification are indicative of the level of those skilled in the art to which this invention pertains. All publications, patents, and patent applications are herein incorporated by reference to the same extent as if each individual publication or patent application was specifically and individually incorporated by reference.

Although the foregoing invention has been described in some detail by way of illustration and example for purposes of clarity of understanding, it will be obvious that certain changes and modifications may be practiced within the scope of the appended claims.

1. A method of determining disease classification in a subject comprising
(a) obtaining a peripheral blood monocyte sample from said subject;
(b) obtaining a synovial fluid monocyte sample from said subject;
(c) assaying the expression level of a nucleotide sequence of interest in said peripheral blood monocyte sample and said synovial fluid monocyte sample; and
(d) comparing expression levels of said nucleotide sequence of interest to a standard expression pattern to determine disease classification.

2. The method of claim 1, wherein said nucleotide sequence of interest is selected from the group consisting of:
(a) nucleic acid molecules having a nucleotide sequence set forth in SEQ ID NOS:1, 3, 5, 7, 9, 11, 13, 15, 17, 19, 21, 23, 25, 27, 29, 31, 33, 35, 37, 38, 39, 40, 41, 42, 43, 44, 45, 46, 47, 48, 49, 50, 51, 52, 53, 54, and 55;
(b) nucleic acid molecules having a nucleotide sequence at least 90% identity to a nucleotide sequence set forth in SEQ ID NOS:1, 3, 5, 7, 9, 11, 13, 15, 17, 19, 21, 23, 25, 27, 29, 31, 33, 35, 37, 38, 39, 40, 41, 42, 43, 44, 45, 46, 47, 48, 49, 50, 51, 52, 53, 54 and 55; and
(c) nucleic acid molecules having a nucleotide sequence that encodes a polypeptide having an amino acid sequence set forth in SEQ ID NOS:2, 4, 6, 8, 10, 12, 14, 16, 18, 20, 22, 24, 26, 28, 30, 32, 34, 36, and 56; and
(d) nucleic acid molecules having a nucleotide sequence that encodes a polypeptide having an amino acid sequence having at least 90% identity to an amino acid set forth in SEQ ID NOS:2, 4, 6, 8, 10, 12, 14, 16, 18, 20, 22, 24, 26, 28, 30, 32, 34, 36, and 56.

3. The method of claim 1, wherein said subject is a mammal.

4. The method of claim 3, wherein said mammal is a human.

5. The method of claim 1, further comprising isolating RNA from said samples.

6. The method of claim 1, wherein assaying said expression levels analyzes the polypeptide encoded by the nucleotide sequence of interest.

7. The method of claim 1, wherein said subject exhibits a juvenile arthritis.

8. The method of claim 1, wherein said disease classification is classification of a juvenile arthritis.

9. The method of claim 8, wherein said juvenile arthritis is selected from the group consisting of pauciarticular juvenile arthritis, polyarticular juvenile arthritis, systemic onset juvenile rheumatoid arthritis, and juvenile onset spondyloarthropathy.

10. A method of determining juvenile arthritis classification in a subject exhibiting juvenile arthritis comprising:
(a) obtaining a peripheral blood monocyte sample from said subject;
(b) obtaining a synovial fluid monocyte sample from said subject;
(c) assaying the expression level of a nucleotide sequence of interest in said peripheral blood monocyte sample and said synovial fluid monocyte sample; and
(d) comparing the expression level of said nucleotide sequence of interest to a standard expression pattern to determine disease classification.

11. The method of claim 10, wherein said nucleotide sequence of interest is selected from the group consisting of:
(a) nucleic acid molecules having a nucleotide sequence set forth in SEQ ID NOS:1, 3, 5, 7, 9, 11, 13, 15, 17, 19, 21, 23, 25, 27, 29, 31, 33, 35, 37, 38, 39, 40, 41, 42, 43, 44, 45, 46, 47, 48, 49, 50, 51, 52, 53, 54, and 55;
(b) nucleic acid molecules having a nucleotide sequence at least 90% identity to a nucleotide sequence set forth in SEQ ID NOS:1, 3, 5, 7, 9, 11, 13, 15, 17, 19, 21, 23, 25, 27, 29, 31, 33, 35, 37, 38, 39, 40, 41, 42, 43, 44, 45, 46, 47, 48, 49, 50, 51, 52, 53, 54, and 55;
(c) nucleic acid molecules having a nucleotide sequence that encodes a polypeptide having an amino acid sequence set forth in SEQ ID NOS:2, 4, 6, 8, 10, 12, 14, 16, 18, 20, 22, 24, 26, 28, 30, 32, 34, 36, and 56; and
(d) nucleic acid molecules having a nucleotide sequence that encodes a polypeptide having an amino acid sequence having at least 90% identity to an amino acid set forth in SEQ ID NOS:2, 4, 6, 8, 10, 12, 14, 16, 18, 20, 22, 24, 26, 28, 30, 32, 34, 36, and 56.

12. The method of claim 10, wherein expression levels of at least five nucleotide sequences of interest are compared to a standard expression pattern.

13. The method of claim 10, wherein expression levels of at least 10 nucleotide sequences of interest are compared to a standard expression pattern.

14. The method of claim 10, wherein expression levels of at least 15 nucleotide sequences of interest are compared to a standard expression pattern.

15. The method of claim 10, wherein expression levels of at least 18 nucleotide sequences of interest are compared to a standard expression pattern.

16. The method of claim 10, wherein said subject is a mammal.

17. The method of claim 16, wherein said mammal is a human.

18. The method of claim 10, wherein said juvenile arthritis is selected from the group consisting of pauciarticular juvenile arthritis, polyarticular juvenile arthritis, systemic onset juvenile rheumatoid arthritis, and juvenile onset spondyloarthropathy.

19. A kit for performing the method of claim 10 comprising:
(a) a peripheral blood monocyte sample collection reagent;
(b) a synovial fluid monocyte sample collection reagent; and
(c) a detection reagent for a nucleotide sequence of interest.

20. The kit of claim 19 comprising detection reagents for at least 18 nucleotide sequences of interest.

21. The method of claim 10, wherein said standard expression pattern is a juvenile arthritis expression pattern.

22. A method of analyzing disease progression in a subject exhibiting juvenile arthritis comprising:
(a) obtaining a first peripheral blood monocyte sample from said subject;
(b) obtaining a first synovial fluid monocyte sample from said subject;
(c) assaying a first expression level of a nucleotide sequence of interest in said first peripheral blood monocyte sample and said first synovial fluid monocyte sample;
(d) obtaining a second peripheral blood monocyte sample from said subject;
(e) obtaining a second synovial fluid monocyte sample from said subject;
(f) assaying a second expression level of a nucleotide sequence of interest in said second peripheral blood monocyte sample and said second synovial fluid monocyte sample; and
(g) comparing the first and second expression levels of said nucleotide sequence of interest.

23. A kit for performing the method of claim 22 comprising:
(a) a peripheral blood monocyte sample collection reagent;
(b) a synovial fluid monocyte sample collection reagent; and
(c) a detection reagent for a nucleotide sequence of interest.

24. A method of identifying a nucleotide sequence of interest expression modulating compound comprising the steps of:
(a) obtaining a first peripheral blood monocyte sample from a subject exhibiting juvenile arthritis;
(b) obtaining a first synovial fluid monocyte sample from said subject;
(c) assaying a first expression level of a nucleotide sequence of interest in said first peripheral blood monocyte sample and said first synovial fluid monocyte sample;
(d) administering a compound of interest;
(e) obtaining a second peripheral blood monocyte sample;
(f) obtaining a second synovial fluid monocyte sample;
(g) assaying a second expression level of a nucleotide sequence of interest in said second peripheral blood monocyte sample and said second synovial fluid monocyte sample; and
(h) comparing the first and second expression levels of said nucleotide sequence of interest.

25. The method of claim 24, wherein said subject is selected from the group consisting of human, mouse, rabbit, dog, pig, goat, cow, rat, monkey, chimpanzee, and sheep.

26. The method of claim 24, wherein said compound of interest is administered to said subject, to cells obtained from said subject, or to cells cultured from said subject.

27. A method of identifying an arthritis modulating compound comprising the steps of:
(a) obtaining a first peripheral blood monocyte sample from a subject exhibiting juvenile arthritis;
(b) obtaining a first synovial fluid monocyte sample from said subject;
(c) assaying a first expression level of a nucleotide sequence of interest in said first peripheral blood monocyte sample and said first synovial fluid monocyte sample;
(d) administering a compound of interest;
(e) obtaining a second peripheral blood monocyte sample;
(f) obtaining a second synovial fluid monocyte sample;
(g) assaying a second expression level of a nucleotide sequence of interest in said second peripheral blood monocyte sample and said second synovial fluid monocyte sample; and
(h) comparing the first and second expression levels of said nucleotide sequence of interest.

28. An arthritis modulating compound identified by the method of claim 27.

29. The method of claim 27, wherein said compound of interest is administered to said subject, to cells obtained from said subject, or to cells cultured from said subject.

30. A method of determining juvenile arthritis classification in a subject exhibiting juvenile arthritis comprising the steps of:
(a) obtaining one or more biological samples from the subject; and
(b) assaying an expression pattern of CXCL chemokines in the biological samples to determine juvenile arthritis classification of the subject.

31. The method of claim 30, wherein said biological sample is a peripheral blood monocyte sample.

32. The method of claim 30, wherein said biological sample is a synovial fluid monocyte sample.

33. The method of claim 30, wherein multiple biological samples are obtained.

34. The method of claim 33, wherein said multiple biological samples comprise a peripheral blood monocyte sample and a synovial fluid monocyte sample.

35. The method of claim 33, wherein said multiple biological samples are obtained at multiple time points.

36. A method of determining juvenile arthritis progression in a subject exhibiting juvenile arthritis comprising the steps of:
(a) obtaining one or more biological samples from the subject; and
(b) assaying an expression pattern of CXCL chemokines in the biological samples to determine juvenile arthritis progression in the subject.

37. A method of determining disease classification in a subject comprising
(a) obtaining a peripheral blood monocyte sample from said subject;
(b) assaying the expression level of a nucleotide sequence of interest in said peripheral blood monocyte sample; and
(c) comparing said expression level of said nucleotide sequence of interest to a standard expression pattern to determine disease classification.

38. A method of determining disease classification in a subject comprising
(a) obtaining a synovial fluid monocyte sample from said subject;
(b) assaying the expression level of a nucleotide sequence of interest in said synovial fluid monocyte sample; and
(c) comparing expression levels of said nucleotide sequence of interest to a standard expression pattern to determine disease classification.

39. A method of determining juvenile arthritis classification in a subject exhibiting juvenile arthritis comprising:
(a) obtaining a peripheral blood monocyte sample from said subject;
(b) assaying the expression level of a nucleotide sequence of interest in said peripheral blood monocyte sample; and
(c) comparing said expression level of said nucleotide sequence of interest to a standard expression pattern to determine disease classification.

40. A kit for performing the method of claim 39 comprising:
(a) a peripheral blood monocyte sample collection reagent; and
(b) a detection reagent for a nucleotide sequence of interest.

41. A method of determining juvenile arthritis classification in a subject exhibiting juvenile arthritis comprising:
(a) obtaining a synovial fluid monocyte sample from said subject;
(b) assaying the expression level of a nucleotide sequence of interest in said synovial fluid monocyte sample; and
(c) comparing said expression level of said nucleotide sequence of interest to a standard expression pattern to determine disease classification.

42. A kit for performing the method of claim 41 comprising:
(a) a synovial fluid monocyte sample collection reagent; and
(b) a detection reagent for a nucleotide sequence of interest.

43. A method of analyzing disease progression in a subject exhibiting juvenile arthritis comprising:
(a) obtaining a first peripheral blood monocyte sample from said subject;
(b) assaying a first expression level of a nucleotide sequence of interest in said first peripheral blood monocyte sample;
(c) obtaining a second peripheral blood monocyte sample from said subject;
(d) assaying a second expression level of a nucleotide sequence of interest in said second peripheral blood monocyte sample; and
(e) comparing said first and second expression levels of said nucleotide sequence of interest.

44. A method of analyzing disease progression in a subject exhibiting juvenile arthritis comprising:
(a) obtaining a first synovial fluid monocyte sample from a subject exhibiting juvenile arthritis;
(b) assaying a first expression level of a nucleotide sequence of interest in said first synovial fluid monocyte sample;
(c) obtaining a second synovial fluid monocyte sample from said subject;
(d) assaying a second expression level of a nucleotide sequence of interest in said second synovial fluid monocyte sample; and
(e) comparing said first and second expression levels of said nucleotide sequence of interest.

45. A method of identifying a nucleotide sequence of interest expression modulating compound comprising the steps of:
(a) obtaining a first peripheral blood monocyte sample from a subject exhibiting juvenile arthritis;
(b) assaying a first expression level of a nucleotide sequence of interest in said first peripheral blood monocyte sample;
(c) administering a compound of interest;
(d) obtaining a second peripheral blood monocyte sample;
(e) assaying a second expression level of a nucleotide sequence of interest in said second peripheral blood monocyte sample; and
(f) comparing said first and second expression levels of said nucleotide sequence of interest.

46. A method of identifying a nucleotide sequence of interest expression modulating compound comprising the steps of:
(a) obtaining a first synovial fluid monocyte sample from a subject exhibiting juvenile arthritis;
(b) assaying a first expression level of a nucleotide sequence of interest in first synovial fluid monocyte sample;
(c) administering a compound of interest;
(d) obtaining a second synovial fluid monocyte sample;
(e) assaying a second expression level of a nucleotide sequence of interest in said second synovial fluid monocyte sample; and
(f) comparing said first and second expression levels of said nucleotide sequence of interest.

47. A method of identifying an arthritis modulating compound comprising the steps of:
(a) obtaining a first peripheral blood monocyte sample from a subject exhibiting juvenile arthritis;
(b) assaying a first expression level of a nucleotide sequence of interest in said first peripheral blood monocyte sample;
(c) administering a compound of interest;
(d) obtaining a second peripheral blood monocyte sample;
(e) assaying a second expression level of a nucleotide sequence of interest in said second peripheral blood monocyte sample; and
(f) comparing said first and second expression levels of said nucleotide sequence of interest.

48. A method of identifying an arthritis modulating compound comprising the steps of:
(a) obtaining a first synovial fluid monocyte sample from a subject exhibiting juvenile arthritis;
(b) assaying a first expression level of a nucleotide sequence of interest in first synovial fluid monocyte sample;
(c) administering a compound of interest;
(d) obtaining a second synovial fluid monocyte sample;
(e) assaying a second expression level of a nucleotide sequence of interest in said second synovial fluid monocyte sample; and
(f) comparing said first and second expression levels of said nucleotide sequence of interest.
